SEQUENCE LISTING (28 sequences)

SEQ ID NO: 1 - DNA, 201 bp, Citrullus lanatus
  source 1..201: mol_type = genomic DNA
atcaggtgagtttagatatgtttttctaggtgtttggtctaatagtttaaattttatgtttttaatttattagatttgttgttaaaatacactcaaaaagnagtagatttggtaatttggcaatttacccaaactagaagagttctagtcattttctaaaaacacaatcttattttagccatttatcccgttaacacaatt

SEQ ID NO: 2 - DNA, 201 bp, Citrullus lanatus
  source 1..201: mol_type = genomic DNA
tttatgatatgggagaagaaaaagagggagaaaaaagctcattgggatcagaaaagaaggaaaggaggaggaagaagagtttgtttgaatggagtaaaggngaagaggtaaatctagtgaagaagctgattgagttccgtacaaagaagatgggtgacgaagaattttatccgtttttgagaaacgggtcattggctgagg

SEQ ID NO: 3 - DNA, 26 bp, synthetic construct
  source 1..26: mol_type = other DNA
ggtgagtttagatatgtttttctagg

SEQ ID NO: 4 - DNA, 24 bp, synthetic construct
  source 1..24: mol_type = other DNA
attgtgttaacgggataaatggct

SEQ ID NO: 5 - DNA, 32 bp, synthetic construct
  source 1..32: mol_type = other DNA
cactcaaaaagaagtagatttggtaatttggg

SEQ ID NO: 6 - DNA, 24 bp, synthetic construct
  source 1..24: mol_type = other DNA
atcagaaaagaaggaaaggaggag

SEQ ID NO: 7 - DNA, 24 bp, synthetic construct
  source 1..24: mol_type = other DNA
ctttgtacggaactcaatcagctt

SEQ ID NO: 8 - DNA, 30 bp, synthetic construct
  source 1..30: mol_type = other DNA
tgtttgaatggagtaaaggagaagaggtat

SEQ ID NO: 9 - DNA, 21 bp, synthetic construct
  source 1..21: mol_type = other DNA
attgccccaagcacagagata

SEQ ID NO: 10 - DNA, 24 bp, synthetic construct
  source 1..24: mol_type = other DNA
aaattggtataactgtggcaagaa

SEQ ID NO: 11 - DNA, 30 bp, synthetic construct
  source 1..30: mol_type = other DNA
tagatctctcaacacactttgcaatgcaat

SEQ ID NO: 12 - DNA, 23 bp, synthetic construct
  source 1..23: mol_type = other DNA
tttgagtggttaccgaacattct

SEQ ID NO: 13 - DNA, 23 bp, synthetic construct
  source 1..23: mol_type = other DNA
tctttgtatgctcacgaatttca

SEQ ID NO: 14 - DNA, 30 bp, synthetic construct
  source 1..30: mol_type = other DNA
ttgttaaactaatcggtgaaatttcaaagc

SEQ ID NO: 15 - DNA, 24 bp, synthetic construct
  source 1..24: mol_type = other DNA
tttgctttttcaaattaccactaa

SEQ ID NO: 16 - DNA, 20 bp, synthetic construct
  source 1..20: mol_type = other DNA
tttttcttttggaagccgaa

SEQ ID NO: 17 - DNA, 27 bp, synthetic construct
  source 1..27: mol_type = other DNA
aaaaacctccccttctgatttgatctg

SEQ ID NO: 18 - DNA, 24 bp, synthetic construct
  source 1..24: mol_type = other DNA
ttatcaggattatttgcactgaag

SEQ ID NO: 19 - DNA, 24 bp, synthetic construct
  source 1..24: mol_type = other DNA
atagctttcacaagttttggctct

SEQ ID NO: 20 - DNA, 30 bp, synthetic construct
  source 1..30: mol_type = other DNA
ttaaaccatatgcttcctccatcacaaaag

SEQ ID NO: 21 - DNA, 24 bp, synthetic construct
  source 1..24: mol_type = other DNA
tttagagccaaaacttgtgaaagc

SEQ ID NO: 22 - DNA, 24 bp, synthetic construct
  source 1..24: mol_type = other DNA
ttattgctttttgccctaattaca

SEQ ID NO: 23 - DNA, 30 bp, synthetic construct
  source 1..30: mol_type = other DNA
attctgctaattatgttcggaaattgttgc

SEQ ID NO: 24 - DNA, 121 bp, Citrullus lanatus
  source 1..121: mol_type = genomic DNA
cactagctgcgtcattgccccaagcacagagataatttgagtttagatctctcaacacacwttgcaatgcaaaacttgagccttttgttttcttgccacagttataccaatttagttggaa

SEQ ID NO: 25 - DNA, 201 bp, Citrullus lanatus
  source 1..201: mol_type = genomic DNA
tggttaccgaacattctaatttcttctaaaatgacttattttttaaattaagcatttaaaaatatattccaaacaaatcctaaatttttgttaaactaatmggtgaaatttcaaaggttatttatttataggtaacttaaatttgaagagaacatgatattattgaaattcgtgagcatacaaagattattacattgtttt

SEQ ID NO: 26 - DNA, 121 bp, Citrullus lanatus
  source 1..121: mol_type = genomic DNA
ttttgctttttcaaattaccactaaaaaaaaaaaaagaaaaaaagaaaaaaagaaaaaacytccccttctgatttgatctcttcggcttccaaaagaaaaaatgtccaccaaatcccctgt

SEQ ID NO: 27 - DNA, 201 bp, Citrullus lanatus
  source 1..201: mol_type = genomic DNA
attagtactttagaataaaaaagacagaaagaccaagaggatacacttcagttatcaggattatttgcactgaagtgtttattaaaccatatgcttcctcmatcacaaaacatttgtgtcattatttagagccaaaacttgtgaaagctatacatcatgagtgttttcattctgctaattatgttcggaaattgttggagt

SEQ ID NO: 28 - DNA, 121 bp, Citrullus lanatus
  source 1..121: mol_type = genomic DNA
gtcattatttagagccaaaacttgtgaaagctatacatcatgagtgttttcattctgctarttatgttcggaaattgttggagtgtaattagggcaaaaagcaataaactcaaaaggagaa

(19) United States
(12) Patent Application Publication
(10) Pub. No.: US 2025/0228176 A1
(43) Pub. Date: Jul. 17, 2025

(54) WATERMELON PLANTS WITH OPTIMIZED SEED SIZES

(71) Applicant: Enza Zaden Beheer B.V., Enkhuizen (NL)

(72) Inventors: Laura Barham Brown, Gilroy, CA (US); Robert Warren Barham, Salinas, CA (US); Melanie Thomas, Enkhuizen (NL)

(21) Appl. No.: 18/999,356

(22) Filed: Dec. 23, 2024

Related U.S. Application Data
(60) Provisional application No. 63/616,199, filed on Dec. 29, 2023.

(51) Int. Cl. A01H 6/34; A01H 5/08

(52) U.S. Cl. CPC A01H 6/342; A01H 5/08

(57) ABSTRACT

The present disclosure provides, among other things, watermelon characterized by traits useful for production of seedless watermelon fruit and/or watermelon fruit with a commercially desirable spotted phenotype. The present disclosure provides, among other things, QTLs and SNPs useful for production of watermelon characterized by traits for production of seedless fruit and/or fruit having a spotted phenotype.

### CROSS-REFERENCE TO RELATED APPLICATIONS

This application claims the benefit of priority to U.S. Provisional Application No. 63/616,199, filed on Dec. 29, 2023, the entire content of which is hereby incorporated by reference.

### SEQUENCE LISTING

A Sequence Listing in the form of an xml file (entitled “EAB-65201_SL.xml”, created on Mar. 18, 2025, and having a size of 26,820 bytes) is hereby incorporated by reference in its entirety.

### BACKGROUND

All cultivated forms of watermelon belong to the polymorphic species Citrullus lanatus. Citrullus lanatus is a member of the family Cucurbitaceae, which includes melons, pumpkins, squashes, gourds, cucumber, loofah, and many weeds. Cultivated watermelons grow as annual plants with large, broad leaves. The leaves may be lobed or simple and are typically orbicular to triangular-ovate in shape. The flowers are monoecious, producing both male and female flowers. The flesh color of cultivated watermelons can be red, yellow, or white, with ovate to oblong strongly compressed seeds that may be brown or white in color. Fruits may be round to oblong or elliptical with rind colors varying from dark green to yellow, and rind patterning varying widely.

Watermelon is an important and valuable field crop with an array of observable and detectable traits of importance both to watermelon consumers and watermelon growers. Fresh watermelons are consumed in many forms, generally fresh, sliced, or as an ingredient in prepared foods. Traits of importance to consumers include taste, texture, size, rind pattern, and fruit shape. Market analysis has shown that consumers select watermelons based on characteristics including sweetness, firmness of flesh, lack of seeds, and rind pattern. Growers are further interested in high yields, which are impacted, e.g., by disease resistance. Watermelon varieties can demonstrate commercial value based on any such features or combinations thereof. Over 80% of watermelons grown in the United States are triploid seedless varieties, preferred for their ease of consumption, increased yield, and premium market value over diploid seeded varieties.

### SUMMARY

The present disclosure provides, among other things, watermelon characterized by traits useful for production of seedless watermelon fruit, such as small pips and/or a commercially desirable spotted phenotype. The present disclosure provides, among other things, QTLs and SNPs useful for production of watermelon characterized by traits for production of seedless fruit, such as small pips and/or a spotted phenotype. In certain embodiments, methods and compositions provided herein allow for the control of the seed size of diploid, triploid, and/or tetraploid watermelon plants. In certain embodiments, methods and compositions provided herein allow for the control of pip size in seedless watermelon fruit. The present disclosure recognizes that small pip size and/or a spotted phenotype traits are commercially valuable in the production of watermelon. As used herein, the term “watermelon” includes watermelon plants, fruit, seeds, and parts (e.g., stems, leaves, flesh, and/or cells), as well as watermelon varieties and/or lines, unless otherwise expressly or contextually indicated. Pips are the immature ovules present in seedless watermelon fruit, the size of which can impact fruit texture, where consumer preferences favor small pip size. As referred to herein, a spotted phenotype can include both a spotted pattern evident on fruit rind as well as a spotted pattern apparent on, e.g., watermelon plant leaves. Accordingly, the spotted phenotype can impact marketability and consumer preference when present on watermelon rind, but is also evidence in watermelon plants with or without fruit, e.g., by the appearance of the watermelon plant leaves.

In at least certain aspects, the present disclosure provides a watermelon seed, where the watermelon seed: (a) includes at least one spotted phenotype QTL associated with a T allele of SNP 134886 and/or a G allele of SNP 86340; and (b) has a seed size of “ss1,” “ss2,” “ss3,” or “ss2-3”. In at least certain aspects, the present disclosure provides a watermelon seed, where the watermelon seed: (a) includes at least one spotted phenotype QTL associated with a T allele of SNP 134886 and/or a G allele of SNP 86340; and (b) has a 100 SdW between 0.28 g and 10.1 g. In certain embodiments, the 100 SdW is between: (a) 0.28 g and 5.9 g; (b) 0.28 g and 4 g; (c) 0.28 g and 3.2 g; (d) 1.1 g and 5.9 g; (e) 1.1 g and 4 g; or (f) 1.1 g and 3.2 g. In certain embodiments, the 100 SdW is between: (a) 0.7 g and 5.5 g; (b) 0.7 g and 4 g; (c) 0.7 g and 2.9 g; (d) 1.9 g and 5.5 g; (e) 1.9 g and 4 g; or (f) 1.9 g and 2.9 g. In certain embodiments, the 100 SdW is between: (a) 1.2 g and 10.1 g; or (b) 4.4 g and 7.2 g. In certain embodiments, the watermelon seed is diploid, triploid, or tetraploid. In certain aspects, the present disclosure provides a watermelon plant produced from such a watermelon seed and/or a fruit produced therefrom, e.g., where the watermelon fruit has a spotted phenotype. In certain embodiments, the watermelon fruit is triploid and has a 33%, 67%, or 100% spotted phenotype. In certain embodiments, the watermelon seed includes, and/or is produced by a female parent including in its genome: (a) at least one seed size QTL on chromosome 2 associated with a T allele of SNP 34078247; and (b) at least one seed size QTL on chromosome 6 associated with an A allele of SNP 5640046, a T allele of SNP 5655752, an A allele of SNP 5828256, and/or a G allele of SNP 5828333. In certain embodiments, the watermelon seed, and/or the female parent, is homozygous for: (a) a seed size QTL on chromosome 2 associated with a T allele of SNP 34078247; and (b) a seed size QTL on chromosome 6 associated with an A allele of SNP 5640046, a T allele of SNP 5655752, an A allele of SNP 5828256, and/or a G allele of SNP 5828333. In certain embodiments, the watermelon seed includes, or is produced by a female parent including in its genome: (a) at least one T allele of SNP 34078247; and (b) at least one of an A allele of SNP 5640046, a T allele of SNP 5655752, an A allele of SNP 5828256, and a G allele of SNP 5828333. In certain embodiments, the watermelon seed, and/or the female parent, is homozygous for: (a) a T allele of SNP 34078247; and (b) at least one of an A allele of SNP 5640046, a T allele of SNP 5655752, an A allele of SNP 5828256, and a G allele of SNP 5828333. In certain embodiments, the watermelon seed, and/or a female parent from which it was produced: (a) is not homozygous for a seed size QTL on chromosome 2 associated with an A allele of SNP 34078247; and (b) is not homozygous for a seed size QTL on chromosome 6 associated with at least one of a SNP allele selected from a C allele of SNP 5640046, a C allele of SNP 5655752, a C allele of SNP 5828256, or an A allele of SNP 5828333. In certain embodiments, the watermelon seed, and/or a female parent from which it was produced: (a) is not homozygous for an A allele of SNP 34078247; and (b) is not homozygous for at least one SNP allele selected from a C allele of SNP 5640046, a C allele of SNP 5655752, a C allele of SNP 5828256, or an A allele of SNP 5828333. In certain embodiments, the watermelon seed is triploid, and has a triploid hybrid offspring genotype set forth in a table selected from Tables 6A-6AB for SNP 34078247, SNP 5640046, and SNP 5828256. In certain embodiments, the watermelon seed has a genotype corresponding to production of watermelon fruit with ps1 or ps2 pip size. In certain embodiments, seedless watermelon produced from a plant grown from the seed have ps1 or ps2 pips.

In at least certain aspects, the present disclosure provides a method of producing a triploid watermelon seed, the method including crossing: (a) a tetraploid female parent that produces seeds having, and/or is produced from a seed having, a seed size of “ss1,” “ss2,” “ss3,” or “ss2-3”; and (b) a diploid male parent. In at least certain aspects, the present disclosure provides a method of producing a triploid watermelon seed, the method including crossing: (a) a tetraploid female parent that produces seeds having, and/or is produced from a seed having, a seed size of 0.28 g 100 SdW to 10.1 g 100 SdW; and (b) a diploid male parent. In at least certain aspects, the present disclosure provides a method of producing a triploid watermelon seed, the method including crossing: (a) a tetraploid female parent: (i) including at least one spotted phenotype QTL associated with a T allele of SNP 134886 and/or a G allele of SNP 86340; and/or (ii) that produces seeds having, and/or is produced from a seed having, a seed size of “ss1,” “ss2,” “ss3,” or “ss2-3”, and (b) a diploid male parent. In at least certain aspects, the present disclosure provides a method of producing a triploid watermelon seed, the method including crossing: (a) a tetraploid female parent: (i) including at least one spotted phenotype QTL associated with a T allele of SNP 134886 and/or a G allele of SNP 86340; and/or (ii) that produces seeds having, and/or is produced from a seed having, a 100 SdW of 0.28 g to 10.1 g, and (b) a diploid male parent. In certain embodiments, the 100 SdW is between 4.4 g and 7.2 g. In certain embodiments, the diploid male parent: (i) includes at least one spotted phenotype QTL associated with a T allele of SNP 134886 and/or a G allele of SNP 86340; and/or (ii) produces seeds having, and/or is produced from a seed having, a seed size of “ss1,” “ss2,” “ss3,” or “ss2-3”. In at least certain aspects, the present disclosure provides a method of producing a triploid watermelon seed, the method including crossing: (a) a tetraploid female parent: (i) including at least one spotted phenotype QTL associated with a T allele of SNP 134886 and/or a G allele of SNP 86340; and/or (ii) that produces seeds having, and/or is produced from a seed having, a seed size of “ss1,” “ss2,” “ss3,” or “ss2-3”, and (b) a diploid male parent. In at least certain aspects, the present disclosure provides a method of producing a triploid watermelon seed, the method including crossing: (a) a tetraploid female parent that produces seeds having, and/or is produced from a seed having, a seed size of “ss1,” “ss2,” “ss3,” or “ss2-3”; and (b) a diploid male parent. In certain embodiments, the diploid male parent includes at least one spotted phenotype QTL associated with a T allele of SNP 134886 and/or a G allele of SNP 86340. In certain embodiments, the method further includes growing a triploid seed produced by the cross, thereby producing a triploid watermelon plant. In certain embodiments, the triploid watermelon plant produces seedless watermelon fruit having a spotted phenotype. In certain embodiments, the seedless watermelon fruit has a 33%, 67%, or 100% spotted phenotype. In certain embodiments, at least one of the diploid male parent and tetraploid female parent is homozygous for the spotted phenotype QTL associated with a T allele of SNP 134886 and/or a G allele of SNP 86340. In certain embodiments, at least one of the diploid male parent and tetraploid female parent includes at least one T allele of SNP 134886 and/or at least one G allele of SNP 86340. In certain embodiments, at least one of the diploid male parent and tetraploid female parent is homozygous for the T allele of SNP 134886 and/or the G allele of SNP 86340. In certain embodiments, the diploid parent 100 SdW is between: (a) 0.28 g and 5.9 g; (b) 0.28 g and 4 g; (c) 0.28 g and 3.2 g; (d) 1.1 g and 5.9 g; (e) 1.1 g and 4 g; or (f) 1.1 g and 3.2 g. In certain embodiments, the diploid parent 100 SdW is between: (a) 0.7 g and 5.5 g; (b) 0.7 g and 4 g; (c) 0.7 g and 2.9 g; (d) 1.9 g and 5.5 g; (e) 1.9 g and 4 g; or (f) 1.9 g and 2.9 g.

In at least certain aspects, the present disclosure provides a watermelon plant, or part thereof, where the watermelon plant or part thereof includes: (a) at least one seed size QTL on chromosome 2 associated with a T allele of SNP 34078247; and (b) at least one seed size QTL on chromosome 6 associated with an A allele of SNP 5640046, a T allele of SNP 5655752, an A allele of SNP 5828256, and/or a G allele of SNP 5828333, and where the watermelon plant has a spotted phenotype. In at least certain aspects, the present disclosure provides a watermelon plant, or part thereof, where the watermelon plant or part thereof is homozygous for: (a) a seed size QTL on chromosome 2 associated with a T allele of SNP 34078247; and (b) a seed size QTL on chromosome 6 associated with at least one of an A allele of SNP 5640046, a T allele of SNP 5655752, an A allele of SNP 5828256, and/or a G allele of SNP 5828333, and where the watermelon plant has a spotted phenotype. In at least certain aspects, the present disclosure provides a watermelon plant, or part thereof, where the watermelon plant or part thereof includes: (a) at least one T allele of SNP 34078247; and (b) at least one of an A allele of SNP 5640046, a T allele of SNP 5655752, an A allele of SNP 5828256, and a G allele of SNP 5828333. In certain embodiments, the watermelon plant or part thereof is homozygous for: (a) a T allele of SNP 34078247; and (b) at least one of an A allele of SNP 5640046, a T allele of SNP 5655752, an A allele of SNP 5828256, and a G allele of SNP 5828333, and where the watermelon plant has a spotted phenotype. In at least certain aspects, the present disclosure provides a watermelon plant, or part thereof, where the watermelon plant or part thereof: (a) is not homozygous for a seed size QTL on chromosome 2 associated with an A allele of SNP 34078247; and (b) is not homozygous for a seed size QTL on chromosome 6 associated with at least one of a SNP allele selected from a C allele of SNP 5640046, a C allele of SNP 5655752, a C allele of SNP 5828256, or an A allele of SNP 5828333, and where the watermelon plant has a spotted phenotype. In at least certain aspects, the present disclosure provides a watermelon plant, or part thereof, where the watermelon plant, or part thereof: (a) is not homozygous for an A allele of SNP 34078247; and (b) is not homozygous for at least one SNP allele selected from a C allele of SNP 5640046, a C allele of SNP 5655752, a C allele of SNP 5828256, or an A allele of SNP 5828333, and where the watermelon plant has a spotted phenotype. In certain embodiments, the watermelon plant is diploid or tetraploid. In certain embodiments, the watermelon plant or part thereof is diploid and has a diploid genotype set forth in any one of Tables 6A-6AB for SNP 34078247, SNP 5640046, and SNP 5828256. In certain embodiments, the watermelon plant or part thereof is tetraploid and has a tetraploid genotype set forth in any one of Tables 6A-6AB for SNP 34078247, SNP 5640046, and SNP 5828256. In certain embodiments, the watermelon plant produces seeds having a mean, median, or mode 100 seed weight (100 SdW) between 0.28 g and 10.1 g. In certain embodiments, the watermelon plant produces seeds having a mean, median, or mode 100 seed weight (100 SdW) between: (a) 0.28 g and 5.9 g; (b) 0.28 g and 4 g; (c) 0.28 g and 3.2 g; (d) 1.1 g and 5.9 g; (e) 1.1 g and 4 g; or (f) 1.1 g and 3.2 g. In certain embodiments, the watermelon plant produces seeds having a mean, median, or mode 100 seed weight (100 SdW) between: (a) 0.7 g and 5.5 g; (b) 0.7 g and 4 g; (c) 0.7 g and 2.9 g; (d) 1.9 g and 5.5 g; (e) 1.9 g and 4 g; or (f) 1.9 g and 2.9 g. In certain embodiments, the watermelon plant produces seeds having a mean, median, or mode 100 seed weight (100 SdW) between: (a) 1.2 g and 10.1 g; or (b) 4.4 g and 7.2 g. In certain embodiments, the watermelon plant or part thereof is triploid and has a triploid genotype set forth in any one of Tables 6A-6AB for SNP 34078247, SNP 5640046, and SNP 5828256. In certain embodiments, the part is a seed. In certain embodiments, the part is a leaf, pollen, embryo, cotyledon, hypocotyl, root, root tip, anther, pistil, flower, or stem. In certain embodiments, the watermelon plant has is a triploid plant having a 33%, 67%, or 100% spotted phenotype. In certain embodiments, the watermelon plant or part thereof includes at least one spotted phenotype QTL associated with a T allele of SNP 134886 and/or a G allele of SNP 86340. In certain embodiments, the watermelon plant or part thereof includes at least one T allele of SNP 134886 and/or at least one G allele of SNP 86340.

In at least certain aspects, the present disclosure provides a method of producing a triploid watermelon, the method including crossing: (a) a tetraploid female parent including: (i) at least one seed size QTL on chromosome 2 associated with a T allele of SNP 34078247; and (ii) at least one seed size QTL on chromosome 6 associated with an A allele of SNP 5640046, a T allele of SNP 5655752, an A allele of SNP 5828256, and/or a G allele of SNP 5828333. (b) a diploid male parent. In certain embodiments, the diploid male parent includes: (i) at least one seed size QTL on chromosome 2 associated with a T allele of SNP 34078247; and (ii) at least one seed size QTL on chromosome 6 associated with an A allele of SNP 5640046, a T allele of SNP 5655752, an A allele of SNP 5828256, and/or a G allele of SNP 5828333. In certain embodiments, the tetraploid female parent includes: (i) at least one T allele of SNP 34078247; and (ii) at least one of an A allele of SNP 5640046, a T allele of SNP 5655752, an A allele of SNP 5828256, or a G allele of SNP 5828333. In certain embodiments, the diploid male parent includes: (i) at least one T allele of SNP 34078247; and (ii) at least one of an A allele of SNP 5640046, a T allele of SNP 5655752, an A allele of SNP 5828256, or a G allele of SNP 5828333. In certain embodiments, the tetraploid female parent is homozygous for: (i) a T allele of SNP 34078247; and (ii) at least one of an A allele of SNP 5640046, a T allele of SNP 5655752, an A allele of SNP 5828256, or a G allele of SNP 5828333. In certain embodiments, the diploid male parent is homozygous for: (i) a T allele of SNP 34078247; and (ii) at least one of an A allele of SNP 5640046, a T allele of SNP 5655752, an A allele of SNP 5828256, or a G allele of SNP 582833. In at least certain aspects, the present disclosure provides a method of producing a seedless watermelon, the method including crossing: (a) a tetraploid female parent, where the tetraploid female parent: (i) is not homozygous for a seed size QTL on chromosome 2 associated with an A allele of SNP 34078247; and (ii) is not homozygous for a seed size QTL on chromosome 6 associated with at least one of a SNP allele selected from a C allele of SNP 5640046, a C allele of SNP 5655752, a C allele of SNP 5828256, or an A allele of SNP 5828333. (b) a diploid male parent. In certain embodiments, the diploid male parent: (i) is not homozygous for a seed size QTL on chromosome 2 associated with an A allele of SNP 34078247; and (ii) is not homozygous for a seed size QTL on chromosome 6 associated with at least one of a SNP allele selected from a C allele of SNP 5640046, a C allele of SNP 5655752, a C allele of SNP 5828256, or an A allele of SNP 5828333; and In at least certain aspects, the present disclosure provides a method of producing a seedless watermelon, the method including crossing: (a) a tetraploid female parent, where the tetraploid female parent: (i) is not homozygous for an A allele of SNP 34078247; and (ii) is not homozygous for at least one SNP allele selected from a C allele of SNP 5640046, a C allele of SNP 5655752, a C allele of SNP 5828256, or an A allele of SNP 5828333. (b) a diploid male parent. In certain embodiments, the diploid male parent: (i) is not homozygous for an A allele of SNP 34078247; and (ii) is not homozygous for at least one SNP allele selected from a C allele of SNP 5640046, a C allele of SNP 5655752, a C allele of SNP 5828256, or an A allele of SNP 5828333; and In certain embodiments, the tetraploid female parent has a tetraploid female parent genotype set forth in a table selected from Tables 6A-6AB for SNP 34078247, SNP 5640046, and SNP 5828256. In certain embodiments, the diploid male parent has a diploid male parent genotype set forth in a table selected from Tables 6A-6AB for SNP 34078247, SNP 5640046, and SNP 5828256. In certain embodiments, each of the tetraploid female parent and/or the diploid male parent produce seeds having a mean, median, or mode 100 SdW between 0.28 g and 10.1 g. In certain embodiments, each of the tetraploid female parent and/or the diploid male parent produce seeds having a mean, median, or mode 100 SdW between: (a) 0.28 g and 5.9 g; (b) 0.28 g and 4 g; (c) 0.28 g and 3.2 g; (d) 1.1 g and 5.9 g; (e) 1.1 g and 4 g; or (f) 1.1 g and 3.2 g. In certain embodiments, each of the tetraploid female parent and/or the diploid male parent produce seeds having a mean, median, or mode 100 SdW between: (a) 0.7 g and 5.5 g; (b) 0.7 g and 4 g; (c) 0.7 g and 2.9 g; (d) 1.9 g and 5.5 g; (e) 1.9 g and 4 g; or (f) 1.9 g and 2.9 g. In certain embodiments, each of the tetraploid female parent and/or the diploid male parent produce seeds having a mean, median, or mode 100 SdW between: (a) 1.2 g and 10.1 g; or (b) 4.4 g and 7.2 g. In certain embodiments, the method further includes growing a triploid seed produced by crossing the diploid male parent and tetraploid female parent thereby producing a triploid watermelon plant. In certain embodiments, the triploid watermelon plant has a genotype set forth in a table selected from Tables 6A-6AB for SNP 34078247, SNP 5640046, and SNP 5828256. In certain embodiments, the method further includes producing seedless watermelon fruit from the triploid watermelon plant. In certain embodiments, at least one of the diploid male parent and tetraploid female parent has a spotted phenotype. In certain embodiments, at least one of the diploid male parent and tetraploid female parent includes at least one spotted phenotype QTL associated with a T allele of SNP 134886 and/or a G allele of SNP 86340. In certain embodiments, at least one of the diploid male parent and tetraploid female parent includes at least one T allele of SNP 134886 and/or at least one G allele of SNP 86340. In certain embodiments, the watermelon fruit from the triploid watermelon plant has a 33%, 67%, or 100% spotted phenotype.

In at least certain aspects, the present disclosure provides a method of producing a triploid watermelon fruit, the method including crossing a triploid watermelon plant with a diploid watermelon plant, where the diploid watermelon plant is characterized in that has a spotted phenotype and/or at least one spotted phenotype QTL associated with a T allele of SNP 134886 and/or a G allele of SNP 86340 In certain embodiments, the diploid watermelon plant (a) is characterized by a 50% spotted phenotype, and/or is heterozygous for the T allele of SNP 134886 and/or the G allele of SNP 86340; or (b) is characterized by a 100% spotted phenotype, and/or is homozygous for the T allele of SNP 134886 and/or the G allele of SNP 86340. In certain embodiments, the triploid watermelon plant is characterized by a 0% spotted phenotype, and/or is homozygous for an A allele of SNP 134886 and homozygous for an A allele of SNP 86340. In certain embodiments, the triploid watermelon plant is characterized by a 33%, 67%, or 100% spotted phenotype, and/or includes at least one T allele of SNP 134886 and/or at least one G allele of SNP 86340. In at least certain aspects, the present disclosure provides a method of producing a triploid watermelon fruit, the method including crossing a triploid watermelon plant with a diploid watermelon plant, where the triploid watermelon plant is characterized in that has a spotted phenotype and/or at least one spotted phenotype QTL associated with a T allele of SNP 134886 and/or a G allele of SNP 86340, and where the diploid watermelon is characterized by a 0% spotted phenotype, and/or is homozygous for an A allele of SNP 134886 and homozygous for an A allele of SNP 86340. In certain embodiments, the triploid watermelon plant is characterized by a 33%, 67%, or 100% spotted phenotype, and/or includes at least one T allele of SNP 134886 and/or at least one G allele of SNP 86340 In certain embodiments, the triploid watermelon plant has a spotted rind percentage phenotype that differs from the spotted rind percentage phenotype of the diploid watermelon plant.

In at least certain aspects, the present disclosure provides a method of selecting a watermelon plant, or part thereof, for use in production of a seedless watermelon, where selection includes detecting, from a representative sample, that the watermelon plant or part thereof includes: (a) at least one seed size QTL on chromosome 2 associated with a T allele of SNP 34078247; and (b) at least one seed size QTL on chromosome 6 associated with an A allele of SNP 5640046, a T allele of SNP 5655752, an A allele of SNP 5828256, and/or a G allele of SNP 5828333. In at least certain aspects, the present disclosure provides a method of selecting a watermelon plant, or part thereof, for use in production of a seedless watermelon, where selection includes detecting, from a representative sample, that the watermelon plant or part thereof is homozygous for: (a) a seed size QTL on chromosome 2 associated with a T allele of SNP 34078247; and (b) a seed size QTL on chromosome 6 associated with at least one of an A allele of SNP 5640046, a T allele of SNP 5655752, an A allele of SNP 5828256, and/or a G allele of SNP 5828333. In at least certain aspects, the present disclosure provides a method of selecting a watermelon plant, or part thereof, for use in production of a seedless watermelon, where selection includes detecting, from a representative sample, that the watermelon plant or part thereof includes: (a) at least one T allele of SNP 34078247; and (b) at least one of an A allele of SNP 5640046, a T allele of SNP 5655752, an A allele of SNP 5828256, and a G allele of SNP 5828333. In certain embodiments, the watermelon plant, or part thereof, is homozygous for: (a) a T allele of SNP 34078247; and (b) at least one of an A allele of SNP 5640046, a T allele of SNP 5655752, an A allele of SNP 5828256, and a G allele of SNP 5828333. In at least certain aspects, the present disclosure provides a method of selecting a watermelon plant, or part thereof, for use in production of a seedless watermelon, where selection includes detecting, from a representative sample, that the watermelon plant or part thereof: (a) is not homozygous for a seed size QTL on chromosome 2 associated with an A allele of SNP 34078247; and (b) is not homozygous for a seed size QTL on chromosome 6 associated with at least one of a SNP allele selected from a C allele of SNP 5640046, a C allele of SNP 5655752, a C allele of SNP 5828256, or an A allele of SNP 5828333. In at least certain aspects, the present disclosure provides a method of selecting a watermelon plant, or part thereof, for use in production of a seedless watermelon, where selection includes detecting, from a representative sample, that the watermelon plant or part thereof: (a) is not homozygous for an A allele of SNP 34078247; and (b) is not homozygous for at least one SNP allele selected from a C allele of SNP 5640046, a C allele of SNP 5655752, a C allele of SNP 5828256, or an A allele of SNP 5828333. In certain embodiments, the selected watermelon plant, or part thereof, is a diploid watermelon plant and is crossed with a tetraploid watermelon plant, optionally where the tetraploid watermelon plant is plant according to any one of claims 27-42. In certain embodiments, the selected watermelon plant, or part thereof, has a diploid genotype set forth in any one of Tables 6A-6AB for SNP 34078247, SNP 5640046, and SNP 5828256. In certain embodiments, the selected watermelon plant, or part thereof, is a tetraploid watermelon plant and is crossed with a diploid watermelon plant, optionally where the diploid watermelon plant is plant according to any one of claims 27-42. In certain embodiments, the selected watermelon plant, or part thereof, has a tetraploid genotype set forth in any one of Tables 6A-6AB for SNP 34078247, SNP 5640046, and SNP 5828256. In certain embodiments, the selected watermelon plant, or part thereof, is a triploid watermelon plant and is crossed with a pollinizer. In certain embodiments, the selected watermelon plant, or part thereof, is triploid and has a triploid genotype set forth in any one of Tables 6A-6AB for SNP 34078247, SNP 5640046, and SNP 5828256. In certain embodiments, the selected watermelon plant produces seeds having a mean, median, or mode 100 SdW between 0.28 g and 10.1 g. In certain embodiments, the selected watermelon plant produces seeds having a mean, median, or mode 100 SdW between: (a) 0.28 g and 5.9 g; (b) 0.28 g and 4 g; (c) 0.28 g and 3.2 g; (d) 1.1 g and 5.9 g; (e) 1.1 g and 4 g; or (f) 1.1 g and 3.2 g. In certain embodiments, the selected watermelon plant produces seeds having a mean, median, or mode 100 SdW between: (a) 0.7 g and 5.5 g; (b) 0.7 g and 4 g; (c) 0.7 g and 2.9 g; (d) 1.9 g and 5.5 g; (e) 1.9 g and 4 g; or (f) 1.9 g and 2.9 g. In certain embodiments, the selected watermelon plant produces seeds having a mean, median, or mode 100 SdW between: (a) 1.2 g and 10.1 g; or (b) 4.4 g and 7.2 g. In certain embodiments, the selected part is a seed, optionally where a watermelon plant is grown from the selected seed. In certain embodiments, the selected watermelon plant, or a watermelon plant produced from the selected part thereof, has a spotted phenotype. In certain embodiments, the selected watermelon plant, or the watermelon plant produced from the selected part thereof, is a triploid plant having a 33%, 67%, or 100% spotted phenotype. In certain embodiments, the watermelon plant or part thereof includes at least one spotted phenotype QTL associated with a T allele of SNP 134886 and/or a G allele of SNP 86340. In certain embodiments, the watermelon plant or part thereof includes at least one T allele of SNP 134886 and/or at least one G allele of SNP 86340.

In at least certain aspects, the present disclosure provides a watermelon seed designated as ‘E26 S.00185’, representative sample of seed having been deposited under NCIMB Accession Number 44130. In some embodiment, the present disclosure provides a Citrullus lanatus watermelon plant produced by growing ‘E26S.00185’ watermelon seed and/or parts isolated therefrom (which plants and parts can be referred to, e.g., as ‘E26S.00185’ plants and ‘E26S.00185’ parts, respectively). The present disclosure further provides a watermelon plant produced by growing such a seed, as well as a plant part thereof, optionally where the part is a leaf, an ovule, a pollen grain, a fruit, or a cell. In certain embodiments, the part is a fruit. In certain embodiments, the present disclosure further provides a watermelon plant having all, or essentially all, the physiological and morphological characteristics of the watermelon plant produced by growing such a seed, as well as a plant part thereof, optionally where the part is a leaf, an ovule, a pollen grain, a fruit, and/or a cell. In certain embodiments, the part is a fruit. In certain embodiments, the present disclosure further provides a pollen grain and/or an ovule of the plant produced by growing such a seed. In certain embodiments, the present disclosure further provides a protoplast produced from the plant produced by growing such a seed. In certain embodiments, the present disclosure provides a tissue or cell culture produced from protoplasts or cells from the plant produced by growing such a seed, where the cells or protoplasts are produced from a plant part selected from the group consisting of root, root tip, meristematic cell, stem, hypocotyl, petiole, cotyledon, leaf, flower, anther, pollen, pistil, and fruit. In certain embodiments, the present disclosure provides a watermelon plant regenerated from the tissue culture, where the plant has all, or essentially all, of the morphological and physiological characteristics of a watermelon plant produced by growing watermelon seed designated as ‘E26S.00185’, representative sample of seed having been deposited under NCIMB Accession Number 44130. In some embodiments, the present disclosure provides a method of producing seedless watermelon fruit, the method including: (a) crossing the plant produced by growing such a seed with a diploid watermelon plant; (b) allowing seedless fruit to form; and (c) harvesting the seedless fruit.

In at least certain aspects, the present disclosure provides a method of vegetatively propagating a plant of watermelon variety ‘E26S.00185’, the method including the steps of: (a) collecting tissue capable of being propagated from a plant of watermelon variety ‘E26S.00185’, representative seed of the watermelon variety ‘E26S.00185’ having been deposited under NCIMB Accession Number 44130; and (b) producing a rooted plant from the tissue.

In at least certain aspects, the present disclosure provides a watermelon seed designated as ‘E26S.00171’, representative sample of seed having been deposited under NCIMB Accession Number X2. In some embodiment, the present disclosure provides a Citrullus lanatus watermelon plant produced by growing ‘E26S.00171’ watermelon seed and/or parts isolated therefrom (which plants and parts can be referred to, e.g., as ‘E26S.00171’ plants and ‘E26S.00171’ parts, respectively). The present disclosure further provides a watermelon plant produced by growing such a seed, as well as a plant part thereof, optionally where the part is a leaf, an ovule, a pollen grain, a fruit, or a cell. In certain embodiments, the part is a fruit. In certain embodiments, the present disclosure further provides a watermelon plant having all, or essentially all, the physiological and morphological characteristics of the watermelon plant produced by growing such a seed, as well as a plant part thereof, optionally where the part is a leaf, an ovule, a pollen grain, a fruit, and/or a cell. In certain embodiments, the part is a fruit. In certain embodiments, the present disclosure further provides a pollen grain and/or an ovule of the plant produced by growing such a seed. In certain embodiments, the present disclosure further provides a protoplast produced from the plant produced by growing such a seed. In certain embodiments, the present disclosure provides a tissue or cell culture produced from protoplasts or cells from the plant produced by growing such a seed, where the cells or protoplasts are produced from a plant part selected from the group consisting of root, root tip, meristematic cell, stem, hypocotyl, petiole, cotyledon, leaf, flower, anther, pollen, pistil, and fruit. In certain embodiments, the present disclosure provides a watermelon plant regenerated from the tissue culture, where the plant has all, or essentially all, of the morphological and physiological characteristics of a watermelon plant produced by growing watermelon seed designated as ‘E26S.00171’, representative sample of seed having been deposited under NCIMB Accession Number X2. In some embodiments, the present disclosure provides a method of producing seedless watermelon fruit, the method including: (a) crossing the plant produced by growing such a seed with a diploid watermelon plant; (b) allowing seedless fruit to form; and (c) harvesting the seedless fruit.

In at least certain aspects, the present disclosure provides a method of vegetatively propagating a plant of watermelon variety ‘E26S.00171’, the method including the steps of: (a) collecting tissue capable of being propagated from a plant of watermelon variety ‘E26S.00171’, representative seed of the watermelon variety ‘E26S.00171’ having been deposited under NCIMB Accession Number X2; and (b) producing a rooted plant from the tissue.

### Definitions

A, An, The, Or: As used herein, “a”, “an”, and “the” refer to one or to more than one (i.e., to at least one) of the grammatical object of the article. By way of example, “an element” discloses embodiments of exactly one element and embodiments including more than one element. As used herein, the terms “or” and “and/or”, as conjunctions in a list of at least two elements, encompass and disclose embodiments in which the listed elements are included in the alternative, together, or in any combination.

Allele: As used herein, an “allele” is any of one or more alternative forms of a genetic element (such as a locus, gene, or SNP). A polyploid organism can have one allele for a given genetic element on each corresponding chromosome of its genome. Different alleles and/or combinations of alleles can be associated with one or more phenotypic traits.

Associated with: Two events or entities are “associated” with one another, as that term is used herein, if the presence, level and/or form of one is correlated with that of the other. For example, a particular allele is considered to be associated with a particular genotype if its presence, level and/or form correlates with incidence of and/or susceptibility to the phenotype (e.g., across a relevant population).

Backcrossing. As used herein, “backcrossing” is a breeding practice that refers to the repeated crossing of progeny back to a particular parent (e.g., to plants of a particular parental line, variety, or plant), where each successive cross increases similarity between progeny and the particular parent.

Corresponding to: As used herein, the term “corresponding to” may be used to designate the position/identity of a structural element in a compound or composition through comparison with an appropriate reference compound or composition. Examples of corresponding sequences can include, e.g., two distinct sequences (e.g., chromosomes or portions thereof) that represent the same region of a genome (e.g., the same chromosome or portion of a chromosome). For example, in some embodiments, a SNP may be identified as “corresponding to” a position in the sequence of a chromosome or portion thereof if, based on context, a person of skill in the art would recognize that the SNP and position align. In some embodiments, a QTL may be identified as “corresponding to” a sequence of a chromosome or portion thereof if, based on context, a person of skill in the art would recognize that the QTL and sequence are associated with the same trait, are associated with the same or corresponding SNPs, and/or align. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE

Essentially all the physiological and morphological characteristics: A plant having essentially all the physiological and morphological characteristics of another plant means that the plants share essentially all physiological and morphological characteristics identified herein, except, e.g., where applicable, with respect to characteristics derived from a converted gene that differs between the plants as a result backcrossing, mutation, or genetic engineering.

Genotype: As used herein, the term “genotype” refers to the combination of alleles at corresponding loci of a genome, e.g., in a given cell or organism. For example, the genotype of a diploid subject for a SNP refers to the two alleles of the SNP present in the genome of the organism. The genotype of a diploid subject for a QTL refers to the two alleles of the QTL present in the genome of the organism.

Improve, increase, inhibit, decrease or reduce: As used herein, the terms “improve”, “increase”, “inhibit”, “decrease” and “reduce”, and grammatical equivalents thereof, indicate qualitative or quantitative difference from a reference.

Pips: As used herein, “pips” refer to immature ovules present in fruit produced by triploid watermelon plants.

Plant: As used herein, the term “plant” includes whole plants (e.g., having all elements typical of the plant type at a given stage of growth), plant seeds, plant cells, plant protoplasts, plant cell tissue cultures from which watermelon plants can be regenerated, plant calli, plant clumps, and parts of plants. Parts of plants include, without limitation, leaves, pollen, embryos, cotyledons, hypocotyl, roots, root tips, anthers, pistils, flowers, seeds, stems, and the like.

Quantitative Trait Loci (QTL): As used herein, “quantitative trait loci” (QTL) are genetic loci that control (e.g., have a significant effect on) a phenotypic trait. In some non-limiting examples, a phenotypic trait controlled by a QTL is complex (e.g., encompasses more than two possible phenotypes and/or a continuum of possible phenotypes) and/or is controlled by multiple loci. QTLs can be identified by genotypic traits representative of or associated with the QTL (e.g., one or more SNPs).

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference agent, sample, sequence, subject, animal, or individual, or population thereof, or measure or characteristic representative thereof. In some embodiments, a nucleic as sequence (e.g., a SNP or portion of a chromosome, e.g., a QTL) is compared with a reference sequence.

Single Nucleotide Polymorphism (SNP): As used herein, a “single nucleotide polymorphism” (SNP) refers to a position of a nucleic acid sequence (e.g., a position of a chromosome) at which the nucleotide present can vary between corresponding sequences (e.g., between corresponding sequences of comparable organisms, and/or between a sample and a reference).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the results of high resolution melting curve analysis. Each group of curves shown in different grey shades corresponds to a specific percentage of spot allele, from top to bottom: 100% spot allele, 0% spot allele, 25% spot allele, 33% spot allele, 50% spot allele, 67% spot allele, and 75% spot allele (black circle designates grouping of curves).

FIG. 2 shows cross-sections of a mature fruit showing fruit flesh (left) and fruit rind (right) and leaves of an exemplary diploid watermelon line with 100% spot allele (i.e., two spot alleles). The fruit rind and the leaves (upper and lower surface) are spotted.

FIG. 3 shows cross-sections of mature fruit showing fruit flesh (bottom left and top right) and fruit rind (bottom right and top left) and leaves of an exemplary tetraploid watermelon line with 100% spot allele (i.e., four spot alleles). The fruit rind and the leaves (upper and lower surface) are spotted.

FIG. 4 shows whole and cross-sectioned fruit of watermelon variety ‘E26S.00185’.

FIG. 5 shows the size of pips of seedless fruit of watermelon variety ‘E26S.00185’.

FIG. 6A-6C show aspects of the watermelon variety ‘Harvest Moon’ (U.S. Pat. No. 9,545,065). FIG. 6A shows the spotted rind phenotype of seedless watermelon of Harvest Moon (left) and watermelon variety ‘E26S.00185’ (right). FIG. 6B shows the spotted leaf phenotype of Harvest Moon. FIG. 6C shows the size of pips of seedless fruit of Harvest Moon.

FIGS. 7A-7B show watermelon variety ‘E26S.00171’. FIG. 7A shows whole fruits at maturity of watermelon variety ‘E26S.00171’. FIG. 7B shows longitudinal cross-sections of fruit of watermelon variety ‘E26S.00171’.

### DETAILED DESCRIPTION

The present disclosure provides, among other things, watermelon characterized by traits useful for production of seedless fruit, such as small pips and/or a commercially desirable spotted phenotype. In certain embodiments, the methods and compositions provided herein allow for the control of the seed size of diploid, triploid, or tetraploid watermelon plants. Watermelon are an important commercial product with value tied to consumer interest in specific watermelon characteristics recognized by those of skill in the art. Such characteristics are therefore also highly sought after by watermelon producers. Watermelon characteristics include, for example, texture and visual appeal, with respect to which seedless fruit and attractive rind patterns are among those characteristics that can contribute to and/or drive consumer interest. Despite decades of commercial development, new traits and combinations of traits can further improve watermelon products.

The present disclosure includes, among other things, genotypes (e.g., QTLs and SNPs), phenotypes, and crosses useful in controlling seed size, pip size, and/or a spotted phenotype. Various SNP loci are provided herein as having two possible alternative alleles (e.g., simply for illustration, a C allele or A allele). As will be appreciated by those of skill in the art, the present disclosure therefore provides that embodiments characterizing the presence or absence of one allele also provide reciprocal disclosure relating to the presence or absence of the alternative allele. Moreover, as those of skill in the art will also appreciate, the indicated alleles are identified by the nucleic acid present in one strand of a chromosome at a given locus, but further encompass the presence of a complementary nucleic acid on an opposing strand (e.g., simply for illustration, C paired with G or A paired with T).

### Seedless Watermelon

Production of seedless watermelon is typically achieved through breeding schemes that produce a triploid watermelon. Because triploid watermelon plants are sterile, or substantially sterile, the fruit that they produce includes few, substantially none, or no mature seeds.

The watermelon genome includes 11 chromosomes. Fertile watermelon plants can be somatically diploid (2n; 22 chromosome) or tetraploid (4n; 44 chromosome). Most watermelon plants are monoecious, having both male and female flowers. Triploid watermelon (3n; 33 chromosome) can be produced by a cross between a diploid male parent and a tetraploid female parent. Triploid watermelon can grow and bloom, but do not produce viable gametes. Triploid watermelon are therefore regarded as sterile, and are referred to as seedless watermelon.

Despite being sterile, female flowers of a triploid watermelon plant must be pollinated in order for the plant to produce fruit. Pollen of a triploid plant cannot induce fruit production; rather pollination must be from male flowers of a 2n or 4n watermelon plant. Plants used to pollinate triploid watermelon and induce fruit production can be referred to as “pollinizers”.

Exemplary pollinizers can have characteristics that promote separation of pollinizer fruit from the seedless fruit of triploid plants or has other characteristics that promote production of seedless watermelon fruit. For example, a pollinizer can be a variety that grows small and/or inedible fruit (e.g., that can be easily distinguished from and/or separated from seedless watermelon fruit). In some embodiments, a pollinizer can be a variety that grows fruit that utilizes less space and/or nutrient resources than plants for production of seedless watermelon fruit. In some embodiments, a pollinizer can be a variety that preferentially produces male flowers and/or produces male flowers in a greater ratio, as compared to female flowers, than a triploid plant it is used to pollinate. In some embodiments, a pollinizer can be a variety that requires less space in a field than a triploid plant it is used to pollinate.

Pollinizer plants can be systematically proportioned and/or placed within a field of triploid plants. For example, pollinizer plants can be planted in a 1:2, 1:3, or 1:4 ratio of pollinizer watermelon plants to triploid watermelon plants. In some embodiments, rows of pollinizer plants can be interspersed with rows of triploid plants. For example, pollinizer rows could be ever other row, every third row, or every fourth row with triploid plants in remaining rows. In some embodiments, pollinizer rows are evenly spaced with triploid plant rows. In other embodiments, rows of triploid plants are evenly spaced and pollinizer rows are placed between rows of triploid plants. In some embodiments, pollinizer plants and triploid plants are planted together in the same row, e.g., where pollinizer plants are even spaced with triploid plants, where triploid plants are evenly spaced and pollinizer plants are placed between triploid plants, or where pollinizer plants are co-planted with triploid plants.

Although seedless watermelon fruit include few, substantially none, or no mature seeds, they can include smaller non-viable ovules known as pips. Pip size is one factor that contributes to consumer interest in and/or satisfaction with particular varieties of seedless watermelon. The size of pips in fruit of triploid plants generally correlates with the size of seeds that would be produced by fertile (e.g., 2n or 4n) plants of the same variety and/or expected based on the triploid plant's parentage or genetics, e.g., if the triploid plant were able to produce a population of viable watermelon seeds. Consumers of seedless watermelon are understood to prefer smaller pips, e.g., pips that have minimal impact on texture when consumed with the flesh of the watermelon fruit.

### Spotted Phenotype

As disclosed herein, watermelon can have a variety of genotypic or phenotypic traits that can contribute to use in watermelon production and/or marketability. Pip size traits are among those provided herein. Other such traits can include a spotted phenotype, e.g., as disclosed in US 2023/0000035, which is incorporated herein by reference in its entirety, and in particular with respect to spotted trait genotypes traits and phenotypes.

An example of a spotted phenotype that is known in the art, e.g., from diploid-only heirloom watermelon varieties, was known as “Moon and Stars.” However, heirloom “Moon and Stars” varieties have limited breeding utility, since they are only available as diploid watermelon, and moreover because the “Moon and Stars” trait was understood to be associated with less desired traits including one or more of low fruit flesh color saturation, large seeds, less firm fruit flesh texture, and/or a fibrous or stringy fruit flesh texture.

The present disclosure provides, for example, watermelon having a spotted phenotype in which rinds of mature fruit display at least one yellow spot of a diameter of at least 0.2 cm, and genotypes can that produce such watermelon. In various embodiments, the present disclosure provides watermelon having a spotted phenotype in which rinds display multiple yellow spots of a diameter of at least 0.2 cm that are distributed or scattered (e.g., randomly or semi-randomly) over the rind surface, and genotypes that produce such watermelon. In various embodiments, rind pattern traits provided herein are not associated with, e.g., low fruit flesh color saturation, less firm fruit flesh texture, and/or a fibrous or stringy fruit flesh texture. In various embodiments, watermelon of the present disclosure with a spotted phenotype and/or genotype are characterized by saturated fruit flesh color, firm fruit flesh texture, and/or fruit flesh texture that is not fibrous or stringy.

A spotted phenotype as disclosed herein can be a phenotype of 2n, 3n, or 4n watermelon plants. The present disclosure includes the recognition that a spotted phenotype can reflect gene dosage based on a single locus (a “spotted locus”), where each chromosome carrying an allele of the locus that contributes to a spotted phenotype (a “spotted allele”) has an additive contribution to the spotted phenotype of watermelon plants. Thus, the spotted locus can carry a spotted allele that is additive for the spotted phenotype, or a “non-spotted allele” that does not contribute to a spotted phenotype. According, spotted genotypes and phenotypes of the present disclosure can be grouped into quantified or semi-quantified categories designated as “0%” (no spotted allele in any watermelon), “33%” (1 spotted allele in 3n watermelon), “50%” (1 spotted allele in 2n watermelon or 2 spotted alleles in 4n watermelon), “67%” (2 spotted alleles in 3n watermelon), “75%” (3 spotted alleles in 4n watermelon), or “100%” spotted (homozygous for spotted alleles in any watermelon). Thus, the presence (e.g., density) of spots is determined by the percentage of spotted alleles present. The present disclosure includes that spotted phenotype contributes to marketability of watermelon plants and is a trait having commercial value. It is further appreciated herein that the specific level of spots (e.g., for 3n plants, 33%, 67%, or 100%) can vary with differing markets and consumer preferences, as well as with changes thereof over time as can occur within and/or across individual markets and consumer populations.

The present disclosure further recognizes that, in order to breed 67% and 100% spotted 3n offspring, it is necessary to have a spotted tetraploid, which to the knowledge of the present inventors was not available to the public prior to US 2023/0000035, which is incorporated herein by reference in its entirety and in particular with respect to spotted watermelon phenotypes and genotypes. Moreover, in order to produce a generation of 3n plants that is consistently characterized by a 67% and 100% spotted phenotype, the parental lines of the cross must include a 4n plant that is homozygous for spotted alleles and a 2n plant that does not include spotted alleles to consistently produce 67% spotted offspring, or a 2n plant that is homozygous for spotted alleles to consistently produce 100% spotted offspring.

The present disclosure further includes that the spotted locus is positioned between, includes, and/or is linked with a QTL that corresponds to, or is, a sequence positioned between position 134886 and position 86340 of Chromosome 4 of a ‘Charleston Gray’ genome. In various embodiments, the spotted locus, and whether it is a spotted allele or a non-spotted allele, can be identified by a marker at SNP 134886 and/or by a marker at SNP 86340. In various embodiments, SNP 134886 can be a SNP that corresponds to position 101 of SEQ ID NO: 1 (atcaggtgagtttagatatgtttttctaggtgtttggtctaatagtttaaattttatgtttttaatttattagatttgttgttaaaatacactcaaaaagna gtagatttggtaatttggcaatttacccaaactagaagagttctagtcattttctaaaaacacaatcttattttagccatttatcccgttaacacaatt). In various embodiments, SNP 86340 can be a SNP that corresponds to position 101 of SEQ ID NO: 2 (tttatgatatgggagaagaaaaagagggagaaaaaagctcattgggatcagaaaagaaggaaaggaggaggaagaagagtttgtttgaa tggagtaaaggngaagaggtaaatctagtgaagaagctgattgagttccgtacaaagaagatgggtgacgaagaattttatccgtttttgaga aacgggtcattggctgagg). In certain embodiments, an allele of SNP 134886 can be determined by amplification using a primer comprising SEQ ID NO: 3 (ggtgagtttagatatgtttttctagg), and/or a reverse primer comprising SEQ ID NO: 4 (attgtgttaacgggataaatggct), and/or a probe comprising SEQ ID NO: 5 (cactcaaaaagaagtagatttggtaatttggg). In certain embodiments, an allele of SNP 86340 can be determined by amplification using a forward primer comprising SEQ ID NO: 6 (atcagaaaagaaggaaaggaggag), a reverse primer comprising SEQ ID NO: 7 (ctttgtacggaactcaatcagctt), and/or a probe comprising SEQ ID NO: 8 (tgtttgaatggagtaaaggagaagaggtat).

In various embodiments, the presence of a spotted allele can be identified by a T allele of SNP 134886. In various embodiments, the presence of a non-spotted allele can be identified by an A allele of SNP 134886. In various embodiments, the presence of a spotted allele can be identified by a G allele of SNP 86340. In various embodiments, the presence of a non-spotted allele can be identified by an A allele of SNP 86340. Accordingly, in various embodiments, the present disclosure provides 2n, 3n, and/or 4n plants that included at least one T allele of SNP 134886 and/or at least one G allele of SNP 86340.

In various embodiments, the present disclosure provides a 2n plant that includes one, or at least one, T allele of SNP 134886 and/or one, or at least one, G allele of SNP 86340. In various embodiments, the present disclosure provides a 2n plant that includes two T alleles of SNP 134886 and/or two G alleles of SNP 86340.

In various embodiments, the present disclosure provides a 3n plant that includes one, or at least one, T allele of SNP 134886 and/or one, or at least one, G allele of SNP 86340. In various embodiments, the present disclosure provides a 3n plant that includes two, or at least two, T alleles of SNP 134886 and/or two, or at least two, G alleles of SNP 86340. In various embodiments, the present disclosure provides a 3n plant that includes three T alleles of SNP 134886 and/or three G alleles of SNP 86340.

In various embodiments, the present disclosure provides a 4n plant that includes one, or at least one, T allele of SNP 134886 and/or one, or at least one, G allele of SNP 86340. In various embodiments, the present disclosure provides a 4n plant that includes two, or at least two, T alleles of SNP 134886 and/or two, or at least two, G alleles of SNP 86340. In various embodiments, the present disclosure provides a 4n plant that includes three, or at least three, T alleles of SNP 134886 and/or three, or at least three, G alleles of SNP 86340. In various embodiments, the present disclosure provides a 4n plant that includes four T alleles of SNP 134886 and/or four G alleles of SNP 86340.

The present disclosure further includes plant genotypes, plants, and breeding schemes useful for production of spotted watermelon (e.g., spotted seedless watermelon). In various embodiments, plant genotypes, plants, and breeding schemes disclosed herein are useful for the production of spotted watermelon (e.g., spotted seedless watermelon). In various embodiments, plant genotypes, plants, and breeding schemes disclosed herein are selected to utilize parental genotypes for spotted watermelon that can be consistently maintained and/or reproduced through multiple generations. In various embodiments, plant genotypes, plants, and breeding schemes disclosed herein are selected to provide consistency in production of spotted watermelon, in that produced spotted watermelon demonstrate low variability in genotype and/or phenotype for spotted watermelon.

In exemplary embodiments, the present disclosure includes a breeding scheme for production of spotted watermelon that includes crossing a diploid (2n) male parent and tetraploid (4n) female parent to produce a triploid (3n) plant that can yield spotted, seedless fruit as set forth herein. Particular exemplary embodiments are set forth below and provided in tables 6A-6AB.

In a particular exemplary embodiment, a 2n male parental variety, line, or plant that is homozygous for a T allele of SNP 134886 and homozygous for a G allele of SNP 86340 is crossed with a 4n female parental variety, line, or plant that is homozygous for a T allele of SNP 134886 and homozygous for a G allele of SNP 86340. Accordingly, the cross produces a 3n plant homozygous for a T allele of SNP 134886 and homozygous for a G allele of SNP 86340, which 3n plant has a “100%” spotted phenotype.

In a particular exemplary embodiment, a 2n male parental variety, line, or plant that is homozygous for an A allele of SNP 134886 and homozygous for an A allele of SNP 86340 is crossed with a 4n female parental variety, line, or plant that is homozygous for a T allele of SNP 134886 and homozygous for a G allele of SNP 86340. Accordingly, the cross produces a 3n plant homozygous for a T allele of SNP 134886 and homozygous for a G allele of SNP 86340, which 3n plant has a “67%” spotted phenotype.

In a particular exemplary embodiment, a 2n male parental variety, line, or plant that is homozygous for a T allele of SNP 134886 and homozygous for a G allele of SNP 86340 is crossed with a 4n female parental variety, line, or plant that is homozygous for an A allele of SNP 134886 and homozygous for an A allele of SNP 86340. Accordingly, the cross produces a 3n plant homozygous for a T allele of SNP 134886 and homozygous for a G allele of SNP 86340, which 3n plant has a “33%” spotted phenotype.

In various embodiments, the present disclosure provides watermelon having a spotted genotype and/or spotted phenotype provided herein in combination with a seed size genotype and/or seed size phenotype provided herein. As will be appreciated from the present disclosure, the disclosed spotted genotype is controlled by a locus on chromosome 4 while the disclosed seed size phenotype is controlled by loci on chromosomes 2 and 6. For at least this reason, one of skill in the art, with the present specification in hand, would be readily able to combine a spotted genotype and/or spotted phenotype provided herein with a seed size genotype and/or seed size phenotype provided herein. Moreover, the spotted phenotype can be combined with any genetic background, leaf coloration or pattern, or rind coloration or pattern.

The present disclosure provides watermelon having a spotted genotype and/or spotted phenotype for rinds that display at least one (e.g., multiple) yellow spots of a diameter of at least 0.2 cm (e.g., distributed or scattered over the rind surface) against a non-yellow background color. In various embodiments, a non-yellow background color can be, without limitation, any known watermelon color and rind pattern, including but not limited to dark green, medium green, peacock, gray, dark mottled stripe, and tiger stripe.

### Seed Size

The present disclosure includes the identification of markers useful to identify, select, and/or produce plants that produce seeds of a desired size. The present disclosure relates to watermelon that can be diploid (2n), triploid (3n), or tetraploid (4n). Unless otherwise indicated by context, reference to watermelon will encompass at least 2n, 3n, and 4n plants. Accordingly, as used herein, the term “homozygous” can refer to identity of a given allele at each and every corresponding chromosome of a watermelon (e.g., on 2 chromosome of a 2n plant, 3 chromosome of a 3n plant, or 4 chromosome of a 4n plant). Likewise, a plant that does not have identity of a referenced allele at each and every corresponding chromosome can be referred to as heterozygous (e.g., to provide just one example, a 3n plant comprising two copies of a particular allele for a give locus, and one copy of a different allele for that locus).

The present disclosure includes the recognition that certain combinations of parent phenotypes and/or genotypes (e.g., a 4n female parent having a particular phenotype and/or genotype, or range thereof, crossed with a 2n male parent having a particular phenotype and/or genotype, or range thereof) can be particularly useful in the production of 3n watermelon. In some embodiments, a 4n female parent and/or a 2n male parent has, or is selected for use in a cross at least in part because it has, a certain phenotype and/or genotype, or range thereof.

The sizes of watermelon seeds and watermelon pips are largely, predominantly, essentially, and/or entirely determined by the genetics or characteristics of the producing parents (e.g., the 4n female parent in a cross between a 4n female parent and a 2n male parent, or the 3n parent in a cross between a 3n parent and a pollinizer), with minor or no contribution by the pollinizer.

The sizes of watermelon seeds can be categorized according to phenotype or genotype. Phenotypes of watermelon can be associated with seed size categories designated herein (from smallest to largest) as seed size 1 (ss1 or “micro”), seed size 2 (ss2), seed size 3 (ss3), and seed size 4 (ss4). Likewise, genotypes of watermelon can be associated with seed size categories designated herein (from smallest to largest) as “ss1” (or “micro”), “ss2”, “ss3”, and “ss4”. Accordingly, reference to any such category encompasses, as distinct embodiments, (i) watermelon plants and parts thereof produced from seeds having, and seeds having, a phenotype of a recited designated category as set forth herein; (ii) watermelon plants and parts thereof produced from seeds having, and seeds having, a genotype of a recited designated category as set forth herein; and (iii) watermelon plants and parts thereof produced from seeds having, and seeds having, both a phenotype and a genotype of a recited designated category.

Phenotypes of the categories of “ss1” (or “micro”), “ss2”, “ss3”, and “ss4” are defined by ranges of seed weight, expressed as the weight of 100 seeds (a measure referred to as 100 SdW). Genotypes of the categories of “ss1” (or “micro”), “ss2”, “ss3”, and “ss4” are defined by particular combinations of alleles at three SNPs disclosed herein, noting for completeness that two of three SNPs can optionally be represented by equivalent alternative SNPs set forth herein.

It is therefore to be appreciated herein that any reference to seed size categories as ss1 (or micro), ss2, ss3, and/or ss4 independently encompass and disclose, except where otherwise clear from context, all of (i) watermelon plants and parts thereof produced from seeds having, and seeds having, a phenotype of an indicated category; (ii) watermelon plants and parts thereof produced from seeds having, and seeds having, a genotype of an indicated category; and (iii) watermelon plants and parts thereof produced from seeds having, and seeds having, both a phenotype and a genotype of an indicated category. Moreover, for the avoidance of doubt, to the extent a watermelon may fall within the genotypic category of a given size designation but not the corresponding phenotypic category, or conversely fall within the phenotypic category of the designated size but not the genotypic category, such watermelon will still be understood to fall under the designated size in that each disclosure of a size category encompasses and discloses embodiments of category membership by phenotype alone, category membership by genotype alone, and category membership by both genotype and phenotype. Accordingly, for example, where seed size “ss1” is designated, such disclosure encompasses all watermelon plants and parts thereof produced from seeds having, and seeds having, an “ss1” genotype, watermelon plants and parts thereof produced from seeds having, and seeds having, an “ss1” phenotype, as well as watermelon plants and parts thereof produced from seeds having, and seeds having, both an “ss1” phenotype and an “ss1” genotype.

In various embodiments, “small” seeds refers to seeds that fall into a category selected from ss1 and ss2 by genotype, and/or that fall into a category selected from ss1 and ss2 by phenotype. In various embodiments “medium” seeds refers to seeds that fall into category ss3 by genotype and/or that fall into category ss3 by phenotype. In various embodiments “large” seeds refers to seeds that fall into category ss4 by genotype and/or that fall into category ss4 by phenotype.

The present disclosure further recognizes, without wishing to be bound by any particular scientific theory, that seed size is impacted by the ploidy of the seed. Typically, the higher the ploidy, the larger the seed size. Accordingly, without wishing to be bound by any scientific theory, relative relationships (e.g., rank order) of seed size category genotype and seed size category phenotype disclosed herein can be retained within and across various ploidies (e.g., among 2n watermelon, among 3n watermelon, and among 4n watermelon), but with differing absolute weight values, e.g., as disclosed herein.

Similarly, the present disclosure includes the recognition that pips of fruit produced by 3n watermelon plants having various genotypes, or of various genotypic categories, with respect to seed size alleles and/or QTLs as disclosed herein will have relative sizes consistent with or corresponding to the relative sizes non-pip seeds (e.g., 2n, 3n, and/or 4n non-pip seeds) for the same genotypes or genotypic categories. Accordingly, without wishing to be bound by any scientific theory, relative relationships (e.g., rank order) of pip size category genotype and pip size category phenotype are controlled by the same QTLs and/or SNPs, and abide the same genetic patterns, as seeds, albeit with differing absolute weight values. It is to be appreciated that consistent or corresponding relative pip size does not necessarily suggest a mathematically precise relationship, but rather a trend or rank ordering that mirrors the size of non-pip seeds or genetic patterns identified therefrom and, which can be sufficient for watermelon breeders to rely on when crossing watermelon plants to achieve a desired pip size. As is noted elsewhere herein, smaller pips are regarded as advantageous for marketability of seedless watermelon.

QTLs and/or SNPs that distinguish categories of seed size likewise distinguish categories of pip size. The sizes of watermelon pips can be categorized according to phenotype or genotype. Phenotypes of watermelon can be associated with pip size categories designated herein (from smallest to largest) as pip size 1 (ps1 or “micro”), pip size 2 (ps2), pip size 3 (ps3), and pip size 4 (ps4). Likewise, genotypes of watermelon can be associated with pip size categories designated herein (from smallest to largest) as “ps1” (or “micro”), “ps2”, “ps3”, and “ps4”. Accordingly, reference to any such category encompasses, as distinct embodiments, (i) 3n watermelon plants and parts thereof that produce or include pips having a phenotype of a recited designated category as set forth herein; (ii) 3n watermelon plants and parts thereof that produce or include pips having a genotype of a recited designated category as set forth herein; and (iii) 3n watermelon plants and parts thereof that produce or include pips having both a phenotype and a genotype of a recited designated category.

Phenotypes of the categories of “ps1” (or “micro”), “ps2”, “ps3”, and “ps4” are defined by ranges of pip weight, expressed as the weight of 100 pips (a measure referred to as 100 SdW). Genotypes of the categories of “ps1” (or “micro”), “ps2”, “ps3”, and “ps4” are defined by particular combinations of alleles at three SNPs disclosed herein, noting for completeness that two of three SNPs can optionally be represented by equivalent alternative SNPs set forth herein.

It is therefore to be appreciated herein that any reference to pip size categories as ps1 (or micro), ps2, ps3, and/or ps4 independently encompass and disclose, except where otherwise clear from context, all of (i) 3n watermelon plants and parts thereof that produce or include pips having a phenotype of an indicated category; (ii) 3n watermelon plants and parts thereof that produce or include pips having a genotype of an indicated category; and (iii) 3n watermelon plants and parts thereof that produce or include pips having both a phenotype and a genotype of an indicated category. Moreover, for the avoidance of doubt, to the extent a watermelon may fall within the genotypic category of a given size designation but not the corresponding phenotypic category, or conversely fall within the phenotypic category of the designated size but not the genotypic category, such watermelon will still be understood to fall under the designated size in that each disclosure of a size category encompasses and discloses embodiments of category membership by phenotype alone, category membership by genotype alone, and category membership by both genotype and phenotype. Accordingly, for example, where pip size “ps1” is designated, such disclosure encompasses all 3n watermelon plants and parts thereof that produce or include pips having an “ps1” genotype, 3n watermelon plants and parts thereof that produce or include pips having an “ps1” phenotype, as well as 3n watermelon plants and parts thereof that produce or include pips having both an “ps1” phenotype and an “ps1” genotype.

In various embodiments, “small” pips refers to pips that fall into a category selected from ps1 and ps2 by genotype, and/or that fall into a category selected from ps1 and ps2 by phenotype. In various embodiments “medium” pips refers to pips that fall into category ps3 by genotype and/or that fall into category ps3 by phenotype. In various embodiments “large” pips refers to pips that fall into category ps4 by genotype and/or that fall into category ps4 by phenotype.

Without wishing to be bound by any particular model or theory, the present disclosure includes that large pips can be disfavored by consumers. Without wishing to be bound by any particular model or theory, the present disclosure includes that small and medium pips can be favored by consumers, e.g., as compared to large pips. Without wishing to be bound by any particular model or theory, the present disclosure includes that small and medium pips both confer market advantages, and that such market advantages may be particularly acute with respect to watermelon fruit having small pips, and still more acute with respect to watermelon fruit having ps1 pips.

Moreover, phenotypic seed size categories referred to by the ss1, ss2, ss3, or ss4 designations, except where otherwise indicated by context, expressly encompass disclosure of both (1) a broad range that includes the upper and lower boundaries for seeds of the indicated size, and (2) an exemplary range, that includes boundaries that fall within the broad range. Further, seed sizes referred to by the ss1, ss2, ss3, or ss4 designations (and by the designation ss2-3 spanning seed sizes ss2 and ss3), except where otherwise indicated by context, refer to the size ranges relevant to the, each, and/or any applicable ploidy(ies). The present disclosure recognizes and permits that, in various embodiments, within a given ploidy, the range of seed sizes for two size designations (e.g., ss2 and ss3) can overlap. Any such overlap is not to be understood as reducing the clarity or meaning of the present disclosure, but rather is to be understood as the intended description of applicable categories that can be independently applied. Phenotypic categories ss2 and ss3, and related ss2-3, are among the sizes useful in understanding and applying the present disclosure.

TABLE 1
Phenotypic Categories for Seeds Produced by 2n Watermelon

| | |
|---|---|
| Broad Range | Exemplary Range |

| Seed Size | Min (g) | Max (g) | Min (g) | Max (g) |
|---|---|---|---|---|
| ss1 | 0.28 | 1.1 | 0.7 | 1.1 |
| ss2 | 1.1 | 3.2 | 1.9 | 2.9 |
| ss3 | 2.7 | 5.9 | 3.0 | 5.5 |
| ss2-3 | 1.1 | 5.9 | 1.9 | 5.5 |
| ss4 | 4.9 | 17 | 8.0 | 11.0 |

TABLE 2
Phenotypic Categories for seeds Produced by 4n Watermelon

| | |
|---|---|
| Broad Range | Exemplary Range |

| Seed Size | Min (g) | Max (g) | Min (g) | Max (g) |
|---|---|---|---|---|
| ss1 | 0.28 | 1.2 | | |
| ss2-3 | 1.2 | 10.1 | 4.4 | 7.2 |
| ss4 | 10.1 | >10.1 | | |

Accordingly, for example, where a 2n seed size is indicated as, e.g., ss2, the seed is disclosed as having a sizes that falls within, e.g., the broad range and/or the exemplary range of 100 SdW for seed ss2, and so forth for other indicated seed sizes. As shown in the table of 2n seed sizes, 2n ss1 seeds have a weight of 0.28 g to 1.1 g 100 SdW (e.g., 07 g to 1.1 g 100 SdW); 2n ss2 seeds have a weight of 1.1 g to 3.2 g 100 SdW (e.g., 1.9 g to 2.9 g 100 SdW); 2n ss3 seeds have a weight of 2.7 g to 5.9 g 100 SdW (e.g., 3.0 g to 5.5 g 100 SdW); and 2n ss4 seeds have a weight of 4.9 g to 17 g 100 SdW (e.g., 8.0 g to 11.0 g 100 SdW).

The present disclosure further includes plant genotypes, plants, and breeding schemes useful for production of seedless watermelon (e.g., spotted seedless watermelon). In various embodiments, plant genotypes, plants, and breeding schemes disclosed herein are useful for the production of seedless watermelon (e.g., spotted seedless watermelon) with textural characteristics (and/or visual appeal) considered advantageous by watermelon producers and/or consumers. In various embodiments, plant genotypes, plants, and breeding schemes disclosed herein provide seed size characteristics that facilitate harvesting during production in combination with production of small pips. In various embodiments, plant genotypes, plants, and breeding schemes disclosed herein are selected to utilize parental genotypes for seed size that can be consistently maintained and/or reproduced through multiple generations. In various embodiments, plant genotypes, plants, and breeding schemes disclosed herein are selected to provide consistency in production of seedless watermelon, in that produced seedless watermelon demonstrate low variability in genotype for seed size and/or phenotype for seed size. In exemplary embodiments, the present disclosure includes a breeding scheme for production of seedless watermelon that includes crossing a diploid (2n) male parent and tetraploid (4n) female parent to produce a triploid (3n) plant that can yield seedless fruit as set forth herein.

In various embodiments, a 3n watermelon that produces fruit with pips of a desired or indicated category can be produced by selection of parents that each respectively fall into an indicated category, as set forth below in Table 3. The present disclosure expressly includes crosses of 2n and 4n plants as set forth in Table 3, e.g., to achieve a plant that produces 3n seedless watermelon fruit having a pip size as indicated in the table. Crosses and outcomes indicated in Table 3 reflect information determined through extensive breeding across diverse genetic backgrounds by the present inventors and therefore reflects a summary expected outcomes observed from breeding data and field studies. Identification of pip sizes expected in 3n plants produced by various crosses as set forth in Table 3 demonstrates the present inventors' discovery of the result expected based on such crosses; expected size is not the only plausible outcome, but rather an outcome to be observed from typical field crosses. Accordingly, those of skill in the art will appreciate that the discoveries provided in Table 3 are useful for selecting parents and designing breeding programs in which a particular 3n pip size (e.g., a small and/or medium pip size, e.g., a small pip size) is desired, or where a particular 3n pip size (e.g., a large and/or medium pip size, e.g., a large pip size) is not desired.

TABLE 3
Exemplary Crosses of 2n and 4n Plants to Produce
3n Plants Yielding Fruit with a Desired Pip Size

| | | Expected Pip Size of |
|---|---|---|
| | | Seedless Watermelon of 3n |
| 2n Parent Category | 4n Parent Category | Progeny |
| ss1 | ss1 | Small |
| ss1 | ss2 | Small |
| ss1 | ss3 | Small |
| ss1 | ss4 | Small |
| ss2 | ss1 | Small |
| ss2 | ss2 | Small |
| ss2 | ss3 | Small |
| ss2 | ss4 | Small |
| ss3 | ss1 | Small |
| ss3 | ss2 | Small |
| ss3 | ss3 | Medium |
| ss3 | ss4 | Large |
| ss4 | ss1 | Small |
| ss4 | ss2 | Small |
| ss4 | ss3 | Large |
| ss4 | ss4 | Large |

In various embodiments, the present disclosure includes performing a cross of a 2n parent and 4n parent as set forth in Table 3 that produces a 3n plant expected to produce small pips. In various embodiments, the present disclosure includes performing a cross of a 2n parent and 4n parent as set forth in Table 3 that produces a 3n plant expected to produce small or medium pips. In various embodiments, the present disclosure includes performing a cross of a 2n parent and 4n parent as set forth in Table 3 that produces a 3n plant expected to produce pips that are not large pips.

In certain embodiments, a 2n male parent that produces small seed is crossed with a 4n female parent that produces small, medium, or large seed. In certain embodiments, a 2n male parent that produces ss1 seed is crossed with a 4n female parent that produces small, medium, or large seed. In certain embodiments, a 2n male parent that produces ss2 seed is crossed with a 4n female parent that produces small, medium, or large seed. In certain embodiments, a 2n male parent that produces medium or ss3 seed is crossed with a 4n female parent that produces small or medium seed. In certain embodiments, a 2n male parent that produces large seed is crossed with a 4n female parent that produces small seed. In certain embodiments, a 4n female parent that produces small seed is crossed with a 2n male parent that produces small, medium, or large seed. In certain embodiments, a 4n female parent that produces medium seed is crossed with a 2n male parent that produces small or medium seed. In certain embodiments, a 4n female parent that produces medium seed is crossed with a 2n male parent that produces small seed. In certain embodiments, a 4n female parent that produces large seed is crossed with a 2n male parent that produces small seed.

In certain embodiments, a 2n male parent produced from a small seed is crossed with a 4n female parent produced from a small, medium, or large seed. In certain embodiments, a 2n male parent produced from an ss1 seed is crossed with a 4n female parent produced from a small, medium, or large seed. In certain embodiments, a 2n male parent produced from an ss2 seed is crossed with a 4n female parent produced from a small, medium, or large seed. In certain embodiments, a 2n male parent produced from a medium or ss3 seed is crossed with a 4n female parent produced from a small or medium seed. In certain embodiments, a 2n male parent produced from a large seed is crossed with a 4n female parent produced from a small seed. In certain embodiments, a 4n female parent produced from a small seed is crossed with a 2n male parent produced from a small, medium, or large seed. In certain embodiments, a 4n female parent produced from a medium seed is crossed with a 2n male parent produced from a small or medium seed. In certain embodiments, a 4n female parent produced from a medium seed is crossed with a 2n male parent produced from a small seed. In certain embodiments, a 4n female parent produced from a large seed is crossed with a 2n male parent produced from a small seed.

In certain embodiments, a 2n male parent produced from an ss1 seed is crossed with a 4n female parent produced from an ss1 seed. In certain embodiments, a 2n male parent produced from an ss1 seed is crossed with a 4n female parent produced from an ss2 seed. In certain embodiments, a 2n male parent produced from an ss1 seed is crossed with a 4n female parent produced from an ss3 seed. In certain embodiments, a 2n male parent produced from an ss1 seed is crossed with a 4n female parent produced from an ss4 seed.

In certain embodiments, a 2n male parent produced from an ss2 seed is crossed with a 4n female parent produced from an ss1 seed. In certain embodiments, a 2n male parent produced from an ss2 seed is crossed with a 4n female parent produced from an ss2 seed. In certain embodiments, a 2n male parent produced from an ss2 seed is crossed with a 4n female parent produced from an ss3 seed. In certain embodiments, a 2n male parent produced from an ss2 seed is crossed with a 4n female parent produced from an ss4 seed.

In certain embodiments, a 2n male parent produced from an ss3 seed is crossed with a 4n female parent produced from an ss1 seed. In certain embodiments, a 2n male parent produced from an ss3 seed is crossed with a 4n female parent produced from an ss2 seed. In certain embodiments, a 2n male parent produced from an ss3 seed is crossed with a 4n female parent produced from an ss3 seed.

In certain embodiments, a 2n male parent produced from an ss4 seed is crossed with a 4n female parent produced from an ss1 seed. In certain embodiments, a 2n male parent produced from an ss4 seed is crossed with a 4n female parent produced from an ss2 seed.

In certain embodiments, a 2n male parent produced from an ss3 seed is not crossed with a 4n female parent produced from an ss3 seed. In certain embodiments, a 2n male parent produced from an ss3 seed is not crossed with a 4n female parent produced from an ss4 seed.

In certain embodiments, a 2n male parent produced from an ss4 seed is not crossed with a 4n female parent produced from an ss3 seed. In certain embodiments, a 2n male parent produced from an ss4 seed is not crossed with a 4n female parent produced from an ss4 seed.

In certain embodiments, a 2n male parent that produces ss1 seed is crossed with a 4n female parent that produces ss1 seed. In certain embodiments, a 2n male parent that produces ss1 seed is crossed with a 4n female parent that produces ss2 seed. In certain embodiments, a 2n male parent that produces ss1 seed is crossed with a 4n female parent that produces ss3 seed. In certain embodiments, a 2n male parent that produces ss1 seed is crossed with a 4n female parent that produces ss4 seed.

In certain embodiments, a 2n male parent that produces ss2 seed is crossed with a 4n female parent that produces ss1 seed. In certain embodiments, a 2n male parent that produces ss2 seed is crossed with a 4n female parent that produces ss2 seed. In certain embodiments, a 2n male parent that produces ss2 seed is crossed with a 4n female parent that produces ss3 seed. In certain embodiments, a 2n male parent that produces ss2 seed is crossed with a 4n female parent that produces ss4 seed.

In certain embodiments, a 2n male parent that produces ss3 seed is crossed with a 4n female parent that produces ss1 seed. In certain embodiments, a 2n male parent that produces ss3 seed is crossed with a 4n female parent that produces ss2 seed. In certain embodiments, a 2n male parent that produces ss3 seed is crossed with a 4n female parent that produces ss3 seed.

In certain embodiments, a 2n male parent that produces ss4 seed is crossed with a 4n female parent that produces ss1 seed. In certain embodiments, a 2n male parent that produces ss4 seed is crossed with a 4n female parent that produces ss2 seed.

In certain embodiments, a 2n male parent that produces ss3 seed is not crossed with a 4n female parent that produces ss3 seed. In certain embodiments, a 2n male parent that produces ss3 seed is not crossed with a 4n female parent that produces ss4 seed.

In certain embodiments, a 2n male parent that produces ss4 seed is not crossed with a 4n female parent that produces ss3 seed. In certain embodiments, a 2n male parent that produces ss4 seed is not crossed with a 4n female parent that produces ss4 seed.

In certain embodiments, a 4n female parent is produced from seed having a size of 4 g 100 SdW or less. In certain embodiments, a 2n male parent is produced from seed having a size of 3.3 g 100 SdW or less. In certain embodiments, a 4n female parent produced from seed having a size of 4 g 100 SdW or less is crossed with a 2n male parent produced from seed having a size of 3.3 g 100 SdW or less.

In certain embodiments, a 4n female parent produces seed having a size of 4 g 100 SdW or less. In certain embodiments, a 2n male parent produces seed having a size of 3.3 g 100 SdW or less. In certain embodiments, a 4n female parent that produces seed having a size of 4 g 100 SdW or less is crossed with a 2n male parent that produces seed having a size of 3.3 g 100 SdW or less.

As will be apparent throughout the disclosure, and parents and crosses described herein and above, can optionally include that one or both parents carry at least one spotted phenotype allele. In various embodiments, at least one parent is heterozygous for a spotted phenotype allele. In various embodiments, both parents are heterozygous for a spotted phenotype allele. In various embodiments, at least one parent is homozygous for a spotted phenotype allele. In various embodiments, both parents are homozygous for a spotted phenotype allele. In various embodiments, one parent is heterozygous for a spotted phenotype allele and the other parent is homozygous for a spotted phenotype allele. In various embodiments, the cross produces a 3n plant genetically and/or phenotypically characterized by a 33% spotted phenotype. In various embodiments, the cross produces a 3n plant genetically and/or phenotypically characterized by a 67% spotted phenotype. In various embodiments, the cross produces a 3n plant genetically and/or phenotypically characterized by a 100% spotted phenotype. The present disclosure further includes the discovery of QTLs and SNPs that regulate seed and pip size. Seed and pip size SNPs of the present disclosure are provide in Table 4:

TABLE 4
Summary of Three Seed Size Loci and Corresponding SNPs

| | | | SNP | | |
|---|---|---|---|---|---|
| | | | | | Allele |
| | | SNP Position | | Allele | Associated |
| | | and | | Associated | with |
| | | Equivalent | | with Larger | Smaller |
| QTL | Chromosome | Alternative(s) | Alleles | Seed Size | Seed Size |
| 1 | 2 | 34078247 | T/A | T | A |
| 2 | 6 | 5640046 | C/A | C | A |
| | | 5655752 | C/T | C | T |
| 3 | 6 | 5828256 | C/A | C | A |
| | | 5828333 | A/G | A | G |

As referenced herein, the SNP on chromosome 2 at position 34078247 can be referred to, e.g., as SNP “2: 34078247” or as SNP 34078247.

As referenced herein, the SNP on chromosome 6 at position 5640046 can be referred to, e.g., as SNP “6: 5640046” or as SNP 5640046.

As referenced herein, the SNP on chromosome 6 at position 5655752 can be referred to, e.g., as SNP “6: 5655752” or as SNP 5655752.

As referenced herein, the SNP on chromosome 6 at position 5828256 can be referred to, e.g., as SNP “6: 5828256” or as SNP 5828256.

As referenced herein, the SNP on chromosome 6 at position 5828333 can be referred to, e.g., as SNP “6: 5828333” or as SNP 5828333.

The present disclosure includes the discovery that a SNP at Chromosome 2 position 34078247, characterized by an A nucleotide or a T nucleotide, is associated with seed size and/or associated with a QTL that causes or contributes to seed size. In particular, homozygosity for the A allele of SNP 34078247 is associated with, and/or is associated with a QTL that causes or contributes to, a ss1 seed size. The present disclosure includes that homozygosity of the A allele of SNP 34078247 results in an ss1 phenotype that masks other seed size traits.

The present disclosure includes the discovery that a SNP at Chromosome 6 position 5640046, characterized by a C nucleotide or an A nucleotide, is associated with seed size and/or is associated with a QTL that causes or contributes to seed size. In particular, homozygosity for the C allele of SNP 5640046 is associated with, and/or is associated with a QTL that causes or contributes to, large seed size. In various embodiments, the present disclosure provides watermelon (e.g., 2n, 3n, or 4n watermelon) that are not homozygous for the C allele of SNP 5640046, and/or watermelon (e.g., 2n, 3n, or 4n watermelon) that are homozygous for the C allele of SNP 5640046 but are not homozygous for one or more of (A) the C allele of SNP 5828256, and/or (B) the A allele of SNP 5828333.

The present disclosure includes the discovery that a SNP at Chromosome 6 position 5655752, characterized by a C nucleotide or a T nucleotide, is associated with seed size and/or is associated with a QTL that causes or contributes to seed size. In particular, homozygosity for the C allele of SNP 5655752 contributes to and/or is correlated with large seed size. In various embodiments, the present disclosure provides watermelon (e.g., 2n, 3n, or 4n watermelon) that are not homozygous for the C allele of SNP 5655752, and/or watermelon (e.g., 2n, 3n, or 4n watermelon) that are homozygous for the C allele of SNP 5655752 but are not homozygous for one or more of (A) the C allele of SNP 5828256, and/or (B) the A allele of SNP 5828333.

The present disclosure includes the discovery that a SNP at Chromosome 6 position 5828256, characterized by a C nucleotide or an A nucleotide, is associated with seed size and/or is associated with a QTL that causes or contributes to seed size. In particular, homozygosity for the C allele of SNP 5828256 is associated with, and/or is associated with a QTL that causes or contributes to, large seed size. In various embodiments, the present disclosure provides watermelon (e.g., 2n, 3n, or 4n watermelon) that are not homozygous for the C allele of SNP 5828256, and/or watermelon (e.g., 2n, 3n, or 4n watermelon) that are homozygous for the C allele of SNP 5828256 but are not homozygous for one or more of (A) the C allele of SNP 5640046 and/or (B) the C allele of SNP 5655752.

The present disclosure includes the discovery that a SNP at Chromosome 6 position 5828333, characterized by an A nucleotide or a G nucleotide, is associated with seed size and/or is associated with a QTL that causes or contributes to seed size. In particular, homozygosity for the A allele of SNP 5828333 is associated with, and/or is associated with a QTL that causes or contributes to, large seed size. In various embodiments, the present disclosure provides watermelon (e.g., 2n, 3n, or 4n watermelon) that are not homozygous for the A allele of SNP 5828333, and/or watermelon (e.g., 2n, 3n, or 4n watermelon) that are homozygous for the A allele of SNP 5828333 but are not homozygous for one or more of (A) the C allele of SNP 5640046 and/or (B) the C allele of SNP 5655752.

In various embodiments, the present disclosure provides watermelon (e.g., 2n, 3n, or 4n watermelon) that (A) are homozygous for one or both of the C allele of SNP 5640046 and the C allele of SNP 5655752, and further (B) are not homozygous for one or both of the C allele of SNP 5828256 and the A allele of SNP 5828333.

In various embodiments, the present disclosure provides watermelon (e.g., 2n, 3n, or 4n watermelon) that (A) are homozygous for one or both of the C allele of SNP 5828256 and the A allele of SNP 5828333, and further (B) are not homozygous for one or both of the C allele of SNP 5640046 and the C allele of SNP 5655752.

The present disclosure further recognizes that the C allele of SNP 5640046 and the C allele of SNP 5655752 are linked to each other and to the same seed size QTL, and accordingly, in various embodiments, can be referred to interchangeably, whereby reference to one constitutes a reference to other, and vice versa. Accordingly, in certain embodiments, the C allele of SNP 5640046 and the C allele of SNP 5655752 can be referred to as equivalent SNP alleles. The present disclosure further recognizes that the A allele of SNP 5640046 and the T allele of SNP 5655752 are linked to each other and to the same seed size QTL, and accordingly, in various embodiments, can be referred to interchangeably, whereby reference to one constitutes a reference to other, and vice versa. Accordingly, in certain embodiments, the A allele of SNP 5640046 and the T allele of SNP 5655752 can be referred to as equivalent SNP alleles. In various embodiments, the seed size QTL includes each of, or is positioned between position 5640046 and position 5655752.

The present disclosure further recognizes that the C allele of SNP 5828256 and the A allele of SNP 5828333 are linked to each other and to the same seed size QTL, and accordingly, in various embodiments, can be referred to interchangeably, whereby reference to one constitutes a reference to other, and vice versa. Accordingly, in certain embodiments, the C allele of SNP 5828256 and the A allele of SNP 5828333 can be referred to as equivalent SNP alleles. The present disclosure further recognizes that the A allele of SNP 5828256 and the G allele of SNP 5828333 are linked to each other and to the same seed size QTL, and accordingly, in various embodiments, can be referred to interchangeably, whereby reference to one constitutes a reference to other, and vice versa. Accordingly, in certain embodiments, the A allele of SNP 5828256 and the G allele of SNP 5828333 can be referred to as equivalent SNP alleles. In various embodiments, the seed size QTL includes each of, or is positioned between position 5828256 and position 5828333.

Accordingly, the present disclosure provides that watermelon plants that are homozygous for the A allele of SNP 34078247 produce ss1 seeds. The ss1 phenotype of watermelon plants that are homozygous for the A allele of SNP 34078247 masks any effect from other loci such as SNP 5640046 or SNP 5828256.

The present disclosure provides that watermelon plants that are not homozygous for the A allele of SNP 34078247, are homozygous for the C allele of 5640046, and are homozygous for the C allele of 5828256 produce ss4 seeds. The present disclosure further provides that watermelon plants that are not homozygous for the A allele of SNP 34078247, are not homozygous for the C allele of 5640046, and are not homozygous for the C allele of 5828256 produce ss2 seeds. The present disclosure provides that watermelon plants that are not homozygous for the A allele of SNP 34078247, are not homozygous for the C allele of 5640046, and are homozygous for the C allele of 5828256 produce ss3 seeds. The present disclosure provides that watermelon plants that are not homozygous for the A allele of SNP 34078247, are homozygous for the C allele of 5640046, and are homozygous for the A allele of 5828256 produce ss3 seeds. The present disclosure provides that watermelon plants that are not homozygous for the A allele of SNP 34078247, are homozygous for the C allele of 5640046, and are not homozygous for the C allele of 5828256 are expected to produce ss3 seeds, but may in some embodiments produce other seed size including but not limited to ss4.

For the avoidance of doubt, references to the genetics of seed size herein, above, and throughout likewise disclose the genetics of pip size, as the present disclosure includes the recognition that the same SNPs control the relative sizes of both viable watermelon seeds (seeds) and pips.

Accordingly, the present disclosure further provides that 3n watermelon plants that are homozygous for the A allele of SNP 34078247 produce ps1 pips. The ps1 phenotype of 3n watermelon plants that are homozygous for the A allele of SNP 34078247 masks any effect from other loci such as SNP 5640046 or SNP 5828256.

The present disclosure provides that 3n watermelon plants that are not homozygous for the A allele of SNP 34078247, are homozygous for the C allele of 5640046, and are homozygous for the C allele of 5828256 produce ps4 pips. The present disclosure further provides that 3n watermelon plants that are not homozygous for the A allele of SNP 34078247, are not homozygous for the C allele of 5640046, and are not homozygous for the C allele of 5828256 produce ps2 pips. The present disclosure provides that 3n watermelon plants that are not homozygous for the A allele of SNP 34078247, are not homozygous for the C allele of 5640046, and are homozygous for the C allele of 5828256 produce ps3 pips. The present disclosure provides that 3n watermelon plants that are not homozygous for the A allele of SNP 34078247, are homozygous for the C allele of 5640046, and are homozygous for the A allele of 5828256 produce ps3 pips. The present disclosure provides that 3n watermelon plants that are not homozygous for the A allele of SNP 34078247, are homozygous for the C allele of 5640046, and are not homozygous for the C allele of 5828256 are expected to produce ps3 pips, but may in some embodiments produce other pip size including but not limited to ps4.

In various embodiments, a 3n watermelon that produce fruit with pips of a desired or indicated category can be a 3n plant having, or selected for, a genotype as set forth in Table 5 or a genotype equivalent thereto in accordance with the present specification.

TABLE 5
Exemplary Genotypes and Phenotypes 3n Watermelon

| | Genotype | | | Pip Size | Pip Size |
|---|---|---|---|---|---|
| | 2:34078247 | 6:5640046¹ | 6:5828256² | Category | Class |
| 1 | AAA | CCC | CCC | ps1 | Small |
| 2 | AAA | CCC | CCA | ps1 | Small |
| 3 | AAA | CCC | AAC | ps1 | Small |
| 4 | AAA | CCC | AAA | ps1 | Small |
| 5 | AAA | CCA | CCC | ps1 | Small |
| 6 | AAA | CCA | CCA | ps1 | Small |
| 7 | AAA | CCA | AAC | ps1 | Small |
| 8 | AAA | CCA | AAA | ps1 | Small |
| 9 | AAA | AAC | CCC | ps1 | Small |
| 10 | AAA | AAC | CCA | ps1 | Small |
| 11 | AAA | AAC | AAC | ps1 | Small |
| 12 | AAA | AAC | AAA | ps1 | Small |
| 13 | AAA | AAA | CCC | ps1 | Small |
| 14 | AAA | AAA | CCA | ps1 | Small |
| 15 | AAA | AAA | AAC | ps1 | Small |
| 16 | AAA | AAA | AAA | ps1 | Small |
| 17 | TTT/AAT/TTA³ | CCC | CCC | ps4 | Large |
| 18 | TTT/AAT/TTA | CCC | CCA | ps3 | Medium |
| 19 | TTT/AAT/TTA | CCC | AAC | ps3 | Medium |
| 20 | TTT/AAT/TTA | CCC | AAA | ps3 | Medium |
| 21 | TTT/AAT/TTA | CCA | CCC | ps3 | Medium |
| 22 | TTT/AAT/TTA | CCA | CCA | ps2 | Small |
| 23 | TTT/AAT/TTA | CCA | AAC | ps2 | Small |
| 24 | TTT/AAT/TTA | CCA | AAA | ps2 | Small |
| 25 | TTT/AAT/TTA | AAC | CCC | ps3 | Medium |
| 26 | TTT/AAT/TTA | AAC | CCA | ps2 | Small |
| 27 | TTT/AAT/TTA | AAC | AAC | ps2 | Small |
| 28 | TTT/AAT/TTA | AAC | AAA | ps2 | Small |
| 29 | TTT/AAT/TTA | AAA | CCC | ps3 | Medium |
| 30 | TTT/AAT/TTA | AAA | CCA | ps2 | Small |
| 31 | TTT/AAT/TTA | AAA | AAC | ps2 | Small |
| 32 | TTT/AAT/TTA | AAA | AAA | ps2 | Small |

¹Shown alleles for SNP 6:5640046 are representative of genotypes of equivalent SNPs 6:5640046 and 6:5655752. In particular, the C allele of 6:5640046 is equivalent with the C allele of 6:5655752, and the A allele of 6:5640046 is equivalent with the T allele of 6:5655752.
²Shown alleles for SNP 6:5828256 are representative of genotypes of equivalent SNPs 6:5828256 and 6:5828333. In particular, the C allele of 6:5828256 is equivalent with the A allele of 6:5828333, and the A allele of 6:5828256 is equivalent with the G allele of 6:5828333.
³For the avoidance of doubt, “TTT/AAT/TTA” indicates that a given row of the above table encompasses genotypes in which the alleles at SNP 2:34078247 are TTT, AAT, or TTA.

In various embodiments, the present disclosure includes producing, using and/or growing fruit from a 3n watermelon that produces fruit with small or medium pips. In various embodiments, the present disclosure includes producing, using and/or growing fruit from a 3n watermelon that produces fruit with small pips. In various embodiments, the present disclosure includes producing, using and/or growing fruit from a 3n watermelon that produces fruit with ps1 pips. In various embodiments, the present disclosure includes producing, using and/or growing fruit from a 3n watermelon that produces fruit with ps2 pips. In various embodiments, the present disclosure includes producing, using and/or growing fruit from a 3n watermelon that produces fruit with ps1 or ps2 pips. In various embodiments, the present disclosure includes producing, using and/or growing fruit from a 3n watermelon that produces fruit that do not have ps3 or ps4 pips. In various embodiments, the present disclosure includes producing, using and/or growing fruit from a 3n watermelon that produces fruit that do not have ps4 pips. In various embodiments, the present disclosure includes performing a cross of a 2n parent and 4n parent that produces a 3n plant expected to produce such pips.

In various embodiments, the present disclosure provides 3n watermelon having a ps1 pip size. In various embodiments, 3n watermelon having a ps1 pip size have a genotype in accordance with Table 5, e.g., as set forth any one of rows 1-16 of Table 5, or a genotype equivalent thereto (e.g., represented by alternative and/or equivalent SNPs disclosed herein).

In various embodiments, the present disclosure provides 3n watermelon having a ps2 pip size. In various embodiments, 3n watermelon having a ps2 pip size have a genotype in accordance with Table 5, e.g., as set forth any one of rows 22-24, 26-28, 30, 31, or 32 of Table 5, or a genotype equivalent thereto (e.g., represented by alternative and/or equivalent SNPs disclosed herein).

In various embodiments, the present disclosure provides 3n watermelon having a ps1 or ps2 pip size. In various embodiments, 3n watermelon having a ps1 or ps2 pip size have a genotype in accordance with Table 5, e.g., as set forth any one of rows 1-16, 22-24, 26-28, 30, 31, or 32 of Table 5, or a genotype equivalent thereto (e.g., represented by alternative and/or equivalent SNPs disclosed herein).

In various embodiments, the present disclosure provides 3n watermelon having a ps3 pip size. In various embodiments, 3n watermelon having a ps3 pip size have a genotype in accordance with Table 5, e.g., as set forth any one of rows 18-21, 25, or 29 of Table 5, or a genotype equivalent thereto (e.g., represented by alternative and/or equivalent SNPs disclosed herein).

In various embodiments, the present disclosure provides 3n watermelon having a ps3 pip size. In various embodiments, 3n watermelon having a ps3 pip size have a genotype in accordance with Table 5, e.g., as set forth any one of rows 20, 21, 25, or 29 of Table 5, or a genotype equivalent thereto (e.g., represented by alternative and/or equivalent SNPs disclosed herein).

In various embodiments, the present disclosure provides 3n watermelon that does not have a ps3 pip size. In various embodiments, the present disclosure provides 3n watermelon that does not have a ps3 pip size genotype in accordance with Table 5, e.g., as set forth any one of rows 18-21, 25, or 29 of Table 5, or a genotype equivalent thereto (e.g., represented by alternative and/or equivalent SNPs disclosed herein).

In various embodiments, the present disclosure provides 3n watermelon that does not have a ps3 pip size. In various embodiments, the present disclosure provides 3n watermelon that does not have a ps3 pip size genotype in accordance with Table 5, e.g., as set forth any one of rows 18 or 19, or a genotype equivalent thereto (e.g., represented by alternative and/or equivalent SNPs disclosed herein).

In various embodiments, the present disclosure provides 3n watermelon that does not have a ps4 pip size. In various embodiments, the present disclosure provides 3n watermelon that does not have a ps4 pip size genotype in accordance with Table 5, e.g., as set forth any one of rows row 17 of Table 5, or a genotype equivalent thereto (e.g., represented by alternative and/or equivalent SNPs disclosed herein).

In various embodiments, the present disclosure provides 3n watermelon having a small pip size. In various embodiments, 3n watermelon having a small pip size have a genotype in accordance with Table 5, e.g., as set forth any one of rows 1-16, 22-24, 26-28, 30, 31, or 32 of Table 5, or a genotype equivalent thereto (e.g., represented by alternative and/or equivalent SNPs disclosed herein).

In various embodiments, the present disclosure provides 3n watermelon having a medium pip size. In various embodiments, 3n watermelon having a medium pip size have a genotype in accordance with Table 5, e.g., as set forth any one of rows 18-21, 25, or 29 of Table 5, or a genotype equivalent thereto (e.g., represented by alternative and/or equivalent SNPs disclosed herein).

In various embodiments, the present disclosure provides 3n watermelon having a medium pip size. In various embodiments, 3n watermelon having a medium pip size have a genotype in accordance with Table 5, e.g., as set forth any one of rows 20, 21, 25, or 29 of Table 5, or a genotype equivalent thereto (e.g., represented by alternative and/or equivalent SNPs disclosed herein).

In various embodiments, the present disclosure provides 3n watermelon that does not have a medium pip size. In various embodiments, the present disclosure provides 3n watermelon that does not have a medium pip size genotype in accordance with Table 5, e.g., as set forth any one of rows 18-21, 25, or 29 of Table 5, or a genotype equivalent thereto (e.g., represented by alternative and/or equivalent SNPs disclosed herein).

In various embodiments, the present disclosure provides 3n watermelon that does not have a medium pip size. In various embodiments, the present disclosure provides 3n watermelon that does not have a medium pip size genotype in accordance with Table 5, e.g., as set forth any one of rows 18 or 19 of Table 5, or a genotype equivalent thereto (e.g., represented by alternative and/or equivalent SNPs disclosed herein).

In various embodiments, the present disclosure provides 3n watermelon that does not have a large pip size. In various embodiments, the present disclosure provides 3n watermelon that does not have a large pip size genotype in accordance with Table 5, e.g., as set forth any one of row 17 of Table 5, or a genotype equivalent thereto (e.g., represented by alternative and/or equivalent SNPs disclosed herein).

In various embodiments, the present disclosure provides 3n watermelon that, e.g., are homozygous for an A allele of SNP 2:34078247. In various embodiments, such watermelon are categorized as ps1 watermelon and can have small pips and/or ps1 pips. In various embodiments, the present disclosure provides 3n watermelon that, e.g., are not homozygous for an A allele of SNP 2:34078247, are not homozygous for any allele of SNP 6:5640046 or an equivalent SNP disclosed herein, and are not homozygous for any allele of SNP 6:5828256 or an equivalent SNP disclosed herein. In various embodiments, such watermelon are categorized as ps2 watermelon and can have small pips and/or ps2 pips.

As will be apparent throughout the disclosure, 3n plants described herein and/or above can optionally be produced from parental plants (i.e., a 2n male parent and a 4n female parent) that are each respectively homozygous at each of SNP 2:34078247, SNP 6:5640046 and/or an equivalent SNP disclosed herein, and SNP 6:5828256 and/or an equivalent SNP disclosed herein. In various embodiments, 3n plants described herein and/or above can optionally be produced from parental plants that are stable, fixed, and/or inbred varieties. As will be apparent throughout the disclosure, 3n plants described herein and/or above can optionally include at least one spotted phenotype allele. In various embodiments, a 3n plant described herein and/or above can have one spotted phenotype allele. In various embodiments, a 3n plant described herein and/or above can have two spotted phenotype alleles. In various embodiments, a 3n plant described herein and/or above can have three spotted phenotype alleles. In various embodiments, a 3n plant described herein and/or above can be genetically and/or phenotypically characterized by a 33% spotted phenotype. In various embodiments, a 3n plant described herein and/or above can be genetically and/or phenotypically characterized by a 67% spotted phenotype. In various embodiments, a 3n plant described herein and/or above can be genetically and/or phenotypically characterized by a 100% spotted phenotype.

In various embodiments, the present disclosure provides a population of watermelon characterized by, or predominantly characterized by, one or more of the seed size genotypes provided herein with respect to the alleles of SNP 34078247, the alleles of one or both of SNPs 5640046 and 5655752, and the alleles of one or both of SNPs 5828256 and 5828333 (e.g., by any combination of such alleles provided herein and/or set forth in Tables 6A-6AB). In various embodiments, the present disclosure provides a population of watermelon (e.g., watermelon fruits, e.g., a harvest, box, shipment, or market product) that have or predominantly have (e.g., at least 60%, at least 65%, at least 70%, at least 85%, at least 80%, at least 85%, at least 90%, at least 95%, at least 95%, at least 96%, at least 97%, at least 98%, at least 99%, or 100% of watermelon) one or more of the seed size genotypes provided herein with respect to the alleles of SNP 34078247, the alleles of one or both of SNPs 5640046 and 5655752, and the alleles of one or both of SNPs 5828256 and 5828333 (e.g., by any combination of such alleles provided herein and/or set forth in Tables 6A-6AB). In various embodiments, the present disclosure provides a population of watermelon seeds (e.g., a population or container of seeds for planting, a population or container of seeds for commercial sale, a population of seeds produced by a watermelon plant, or a population of seeds within a watermelon) that have or predominantly have (e.g., at least 60%, at least 65%, at least 70%, at least 85%, at least 80%, at least 85%, at least 90%, at least 95%, at least 95%, at least 96%, at least 97%, at least 98%, at least 99%, or 100% of seeds) one or more of the seed size genotypes provided herein with respect to the alleles of SNP 34078247, the alleles of one or both of SNPs 5640046 and 5655752, and the alleles of one or both of SNPs 5828256 and 5828333 (e.g., by any combination of such alleles provided herein and/or set forth in Tables 6A-6AB). In various embodiments, seed size traits provided herein are not associated with, e.g., low fruit flesh color saturation, less firm fruit flesh texture, and/or a fibrous or stringy fruit flesh texture. In various embodiments, watermelon of the present disclosure with a disclosed seed size phenotype and/or genotype are characterized by saturated fruit flesh color, firm fruit flesh texture, and/or fruit flesh texture that is not fibrous or stringy.

In various embodiments, a population of seeds can be, e.g., at least 10, at least 20, at least 30, at least 40, at least 50, at least 100, at least 500, at least 1,000, or at least 10,000, or more seeds. In various embodiments, a population of seeds refers to a population of seeds that is separate or essentially separate from seeds derived from other plants, plant types, varieties, crosses, fields, and/or other sources. In various embodiments, a population of seeds refers to a population of seeds that is separate or essentially separate from seeds that do not have the one or more of the seed size genotypes provided herein with respect to the alleles of SNP 34078247, the alleles of one or both of SNPs 5640046 and 5655752, and the alleles of one or both of SNPs 5828256 and 5828333 (e.g., by any combination of such alleles provided herein and/or set forth in Tables 6A-6AB).

As will be appreciated by those of skill in the art, SNP alleles can be detected by a wide variety of well-known means, some of which have been used for decades. Examples of techniques useful to identify SNP alleles in samples (e.g., in plants or parts thereof, and/or samples derived from and/or representative of any of the foregoing) include techniques based on sequencing of nucleic acids and techniques that are not based on sequences of nucleic acids. Exemplary techniques that include sequencing of nucleic acids include sanger sequencing, genomic sequence, and/or high-throughput sequence. Examples of techniques that do not include sequencing can utilize, e.g., amplification with allele-specific primers followed by, e.g., product quantification or gel electrophoresis, or, e.g., allele-specific probes. Those of skill in the art will be able to readily utilize techniques of detecting SNP alleles based on genetic information provided herein, including without limitation genomic information relating to SNP alleles provided herein. For the avoidance of doubt, detection of SNP alleles provided herein is equivalent to and/or constitutes detection of QTLs disclosed herein and/or associated therewith. Alleles provided herein with respect to both spotted phenotype SNPs and seed size SNPs are disclosed herein and throughout with reference to a ‘Charleston Gray’ genome. As noted elsewhere herein, the reference genome of ‘Charleston Gray’ is described in Wu et al. Plant Biotechnol J. 2019 December; 17(12):2246-2258. doi: 10.1111/pbi.13136.

Accordingly, in various non-limiting embodiments, a SNP of the present disclosure can be detected using one or more primers set forth, e.g., in Table 12 (see SEQ ID NOs: 9-23). In various non-limiting embodiments, a SNP of the present disclosure can be detected using a pair of primers set forth, e.g., in Table 12 (see SEQ ID NOs: 9-23). In various non-limiting embodiments, a SNP of the present disclosure can be detected using a pair of primers and a probe as set forth, e.g., in Table 12 (see SEQ ID NOs: 9-23). In various embodiments, a SNP of the present disclosure can be detected by the presence of a sequence set forth in Table 13 (SEQ ID NOs: 24-28) or a portion thereof that includes the SNP. In various embodiments, a SNP of the present disclosure can be detected by the presence of a portion of a sequence set forth in Table 13 (SEQ ID NOs: 24-28) that includes the SNP, e.g., where the portion comprises at least 8, at least 10, at least 12, at least 14, at least 16, at least 18, at least 20, at least 25, at least 30, at least 35, at least 40, or at least 50 basepairs of a sequence set forth in Table 13 (SEQ ID NOs: 24-28). For the avoidance of doubt, the present disclosure encompasses that discussion herein of a technique for detection of a SNP allele likewise encompass the use of that technique to determine the absence of the SNP allele where the relevant affirmative readout is not detected.

The present disclosure includes that, in various embodiments, further advantage that can be gained through selection of a particular 2n male parent genotype, e.g., for production of 3n hybrids with a consistent genotype for SNP loci and/or QTLs disclosed herein, e.g., when many 3n seeds are produced from a plurality of crosses between a population of 4n plants having a particular genotype (e.g., a 4n variety having SNP loci and/or QTLs disclosed herein) and a population of 2n plants having a particular genotype (e.g., of a 2n variety having SNP loci and/or QTLs disclosed herein). In certain exemplary crosses, a 4n parent and a 2n parent are each homozygous for each of SNPs 34078247, 5640046, and 5828256. In certain embodiments, both the 4n parent and then 2n parent are homozygous for the A allele of SNP 34078247, such that all 3n offspring produce ps1 pips.

In various exemplary crosses, of a 4n parent and a 2n parent, one of the parents is homozygous for the A allele of SNP 34078247 and one is homozygous for the T allele of SNP 34078247, or both the 4n parent and then 2n parent are homozygous for the T allele of SNP 34078247, such that 3n offspring have pips that are not ps1 pips. In certain embodiments, of a 4n parent and a 2n parent, one of the parents is homozygous for the C allele of SNP 5640046 and one is homozygous for the A allele of SNP 5640046, or both the 4n parent and then 2n parent are homozygous for the A allele of SNP 5640046, such that 3n offspring have pips that are not ps 3 or ps4 pips (i.e., are ps1 or ps2 pips). In certain embodiments, of a 4n parent and a 2n parent, one of the parents is homozygous for the C allele of SNP 5828256 and one is homozygous for the A allele of SNP 5828256, or both the 4n parent and then 2n parent are homozygous for the A allele of SNP 5828256, such that 3n offspring have pips that are not ps3 or ps4 pips (i.e., are ps1 or ps2 pips).

TABLE 6A
Exemplary Cross for Production of 3n Watermelon Plants

| | 4n Female | 2n Male | Hybrid |
|---|---|---|---|
| Representative | Parental | Parental | Offspring |
| Locus | Genotype | Genotype | Genotype |
| 34078247 (T/A) | AAAA | AA | AAA |
| 5640046 (C/A) | (Any) | (Any) | (Any) |
| 5828256 (C/A) | (Any) | (Any) | (Any) |
| Seed/pip size | ss1 | ss1 | ps1 |

TABLE 6B
Exemplary Cross for Production of 3n Watermelon Plants

| | 4n Female | 2n Male | Hybrid |
|---|---|---|---|
| Representative | Parental | Parental | Offspring |
| Locus | Genotype | Genotype | Genotype |
| 34078247 (T/A) | TTTT | AA | TTA |
| 5640046 (C/A) | CCCC | AA | CCA |
| 5828256 (C/A) | CCCC | AA | CCA |
| Seed/pip size | ss4 | ss1 | ps2 |

TABLE 6C
Exemplary Cross for Production of 3n Watermelon Plants

| | 4n Female | 2n Male | Hybrid |
|---|---|---|---|
| Representative | Parental | Parental | Offspring |
| Locus | Genotype | Genotype | Genotype |
| 34078247 (T/A) | TTTT | AA | TTA |
| 5640046 (C/A) | CCCC | AA | CCA |
| 5828256 (C/A) | AAAA | CC | AAC |
| Seed/pip size | ss3 | ss1 | ps2 |

TABLE 6D
Exemplary Cross for Production of 3n Watermelon Plants

| | 4n Female | 2n Male | Hybrid |
|---|---|---|---|
| Representative | Parental | Parental | Offspring |
| Locus | Genotype | Genotype | Genotype |
| 34078247 (T/A) | TTTT | AA | TTA |
| 5640046 (C/A) | CCCC | AA | CCA |
| 5828256 (C/A) | AAAA | AA | AAA |
| Seed/pip size | ss3 | ss1 | ps2 |

TABLE 6E
Exemplary Cross for Production of 3n Watermelon Plants

| | 4n Female | 2n Male | Hybrid |
|---|---|---|---|
| Representative | Parental | Parental | Offspring |
| Locus | Genotype | Genotype | Genotype |
| 34078247 (T/A) | TTTT | AA | TTA |
| 5640046 (C/A) | AAAA | CC | AAC |
| 5828256 (C/A) | CCCC | AA | CCA |
| Seed/pip size | ss3 | ss1 | ps2 |

TABLE 6F
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | TTTT | AA | TTA |
| 5640046 (C/A) | AAAA | CC | AAC |
| 5828256 (C/A) | AAAA | CC | AAC |
| Seed/pip size | ss2 | ss1 | ps2 |

TABLE 6G
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | TTTT | AA | TTA |
| 5640046 (C/A) | AAAA | CC | AAC |
| 5828256 (C/A) | AAAA | AA | AAA |
| Seed/pip size | ss2 | ss1 | ps2 |

TABLE 6H
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | TTTT | AA | TTA |
| 5640046 (C/A) | AAAA | AA | AAA |
| 5828256 (C/A) | CCCC | AA | CCA |
| Seed/pip size | ss3 | ss1 | ps2 |

TABLE 6I
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | TTTT | AA | TTA |
| 5640046 (C/A) | AAAA | AA | AAA |
| 5828256 (C/A) | AAAA | CC | AAC |
| Seed/pip size | ss2 | ss1 | ps2 |

TABLE 6J
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | TTTT | AA | TTA |
| 5640046 (C/A) | AAAA | AA | AAA |
| 5828256 (C/A) | AAAA | AA | AAA |
| Seed/pip size | ss2 | ss1 | ps2 |

TABLE 6K
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | AAAA | TT | AAT |
| 5640046 (C/A) | CCCC | AA | CCA |
| 5828256 (C/A) | CCCC | AA | CCA |
| Seed/pip size | ss1 | ss2 | ps2 |

TABLE 6L
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | AAAA | TT | AAT |
| 5640046 (C/A) | CCCC | AA | CCA |
| 5828256 (C/A) | AAAA | CC | AAC |
| Seed/pip size | ss1 | ss3 | ps2 |

TABLE 6M
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | AAAA | TT | AAT |
| 5640046 (C/A) | CCCC | AA | CCA |
| 5828256 (C/A) | AAAA | AA | AAA |
| Seed/pip size | ss1 | ss2 | ps2 |

TABLE 6N
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | AAAA | TT | AAT |
| 5640046 (C/A) | AAAA | CC | AAC |
| 5828256 (C/A) | CCCC | AA | CCA |
| Seed/pip size | ss1 | ss3 | ps2 |

TABLE 6O
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | AAAA | TT | AAT |
| 5640046 (C/A) | AAAA | CC | AAC |
| 5828256 (C/A) | AAAA | CC | AAC |
| Seed/pip size | ss1 | ss4 | ps2 |

TABLE 6P
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | AAAA | TT | AAT |
| 5640046 (C/A) | AAAA | CC | AAC |
| 5828256 (C/A) | AAAA | AA | AAA |
| Seed/pip size | ss1 | ss3 | ps2 |

TABLE 6Q
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | AAAA | TT | AAT |
| 5640046 (C/A) | AAAA | AA | AAA |
| 5828256 (C/A) | CCCC | AA | CCA |
| Seed/pip size | ss1 | ss2 | ps2 |

TABLE 6R
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | AAAA | TT | AAT |
| 5640046 (C/A) | AAAA | AA | AAA |
| 5828256 (C/A) | AAAA | CC | AAC |
| Seed/pip size | ss1 | ss3 | ps2 |

TABLE 6S
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | AAAA | TT | AAT |
| 5640046 (C/A) | AAAA | AA | AAA |
| 5828256 (C/A) | AAAA | AA | AAA |
| Seed/pip size | ss1 | ss2 | ps2 |

TABLE 6T
Exemplary Cross for Production of 3n Watermelon Plants

| | | | Hybrid |
|---|---|---|---|
| Representative | 4n Female | 2n Male | Offspring |
| Locus | Parental Genotype | Parental Genotype | Genotype |
| 34078247 (T/A) | TTTT | TT | TTT |
| 5640046 (C/A) | CCCC | AA | CCA |
| 5828256 (C/A) | CCCC | AA | CCA |
| Seed/pip size | ss4 | ss2 | ps2 |

TABLE 6U
Exemplary Cross for Production of 3n Watermelon Plants

| Representative | 4n Female | 2n Male | Hybrid |
|---|---|---|---|
| Locus | Parental Genotype | Parental Genotype | Offspring Genotype |
| 34078247 (T/A) | TTTT | TT | TTT |
| 5640046 (C/A) | CCCC | AA | CCA |
| 5828256 (C/A) | AAAA | CC | AAC |
| Seed/pip size | ss3 | ss3 | ps2 |

TABLE 6V
Exemplary Cross for Production of 3n Watermelon Plants

| Representative | 4n Female | 2n Male | Hybrid |
|---|---|---|---|
| Locus | Parental Genotype | Parental Genotype | Offspring Genotype |
| 34078247 (T/A) | TTTT | TT | TTT |
| 5640046 (C/A) | CCCC | AA | CCA |
| 5828256 (C/A) | AAAA | AA | AAA |
| Seed/pip size | ss3 | ss2 | ps2 |

TABLE 6W
Exemplary Cross for Production of 3n Watermelon Plants

| Representative | 4n Female | 2n Male | Hybrid |
|---|---|---|---|
| Locus | Parental Genotype | Parental Genotype | Offspring Genotype |
| 34078247 (T/A) | TTTT | TT | TTT |
| 5640046 (C/A) | AAAA | CC | AAC |
| 5828256 (C/A) | CCCC | AA | CCA |
| Seed/pip size | ss3 | ss3 | ps2 |

TABLE 6X
Exemplary Cross for Production of 3n Watermelon Plants

| Representative | 4n Female | 2n Male | Hybrid |
|---|---|---|---|
| Locus | Parental Genotype | Parental Genotype | Offspring Genotype |
| 34078247 (T/A) | TTTT | TT | TTT |
| 5640046 (C/A) | AAAA | CC | AAC |
| 5828256 (C/A) | AAAA | CC | AAC |
| Seed/pip size | ss2 | ss4 | ps2 |

TABLE 6Y
Exemplary Cross for Production of 3n Watermelon Plants

| Representative | 4n Female | 2n Male | Hybrid |
|---|---|---|---|
| Locus | Parental Genotype | Parental Genotype | Offspring Genotype |
| 34078247 (T/A) | TTTT | TT | TTT |
| 5640046 (C/A) | AAAA | CC | AAC |
| 5828256 (C/A) | AAAA | AA | AAA |
| Seed/pip size | ss2 | ss3 | ps2 |

TABLE 6Z
Exemplary Cross for Production of 3n Watermelon Plants

| Representative | 4n Female | 2n Male | Hybrid |
|---|---|---|---|
| Locus | Parental Genotype | Parental Genotype | Offspring Genotype |
| 34078247 (T/A) | TTTT | TT | TTT |
| 5640046 (C/A) | AAAA | AA | AAA |
| 5828256 (C/A) | CCCC | AA | CCA |
| Seed/pip size | ss3 | ss2 | ps2 |

TABLE 6AA
Exemplary Cross for Production of 3n Watermelon Plants

| Representative | 4n Female | 2n Male | Hybrid |
|---|---|---|---|
| Locus | Parental Genotype | Parental Genotype | Offspring Genotype |
| 34078247 (T/A) | TTTT | TT | TTT |
| 5640046 (C/A) | AAAA | AA | AAA |
| 5828256 (C/A) | AAAA | CC | AAC |
| Seed/pip size | ss2 | ss3 | ps2 |

TABLE 6AB
Exemplary Cross for Production of 3n Watermelon Plants

| Representative | 4n Female | 2n Male | Hybrid |
|---|---|---|---|
| Locus | Parental Genotype | Parental Genotype | Offspring Genotype |
| 34078247 (T/A) | TTTT | TT | TTT |
| 5640046 (C/A) | AAAA | AA | AAA |
| 5828256 (C/A) | AAAA | AA | AAA |
| Seed/pip size | ss2 | ss2 | ps2 |

In various embodiments, a 4n female watermelon and/or 4n female parent is homozygous for a seed size QTL on chromosome 2 associated with a T allele of SNP 34078247 and/or for a T allele of SNP 34078247. In various embodiments, a 4n female watermelon and/or 4n female parent is homozygous for a seed size QTL on chromosome 2 associated with an A allele of SNP 34078247 and/or for an A allele of SNP 34078247. In various embodiments, a 4n female watermelon and/or 4n female parent is homozygous for at least one seed size QTL on chromosome 6 associated with an A allele of SNP 5640046, a T allele of SNP 5655752, an A allele of SNP 5828256, and/or a G allele of SNP 5828333, and/or is homozygous for at least one of an A allele of SNP 5640046, a T allele of SNP 5655752, an A allele of SNP 5828256, and/or a G allele of SNP 5828333. In various embodiments, a 4n female watermelon and/or 4n female parent is homozygous for a seed size QTL on chromosome 6 associated with an A allele of SNP 5640046 and/or is homozygous for an A allele of SNP 5640046. In various embodiments, a 4n female watermelon and/or 4n female parent is homozygous for a seed size QTL on chromosome 6 associated with a T allele of SNP 5655752, and/or is homozygous for a T allele of SNP 5655752. In various embodiments, a 4n female watermelon and/or 4n female parent is homozygous for a seed size QTL on chromosome 6 associated with an A allele of SNP 5828256, and/or is homozygous for an A allele of SNP 5828256. In various embodiments, a 4n female watermelon and/or 4n female parent is homozygous for a seed size QTL on chromosome 6 associated with a G allele of SNP 5828333, and/or is homozygous for a G allele of SNP 5828333. In various embodiments, a 4n female watermelon and/or 4n female parent is homozygous for a seed size QTL on chromosome 2 associated with a T allele of SNP 34078247 and/or for a T allele of SNP 34078247, and is further homozygous for a seed size QTL on chromosome 6 associated with an A allele of SNP 5640046 and/or is homozygous for an A allele of SNP 5640046. In various embodiments, a 4n female watermelon and/or 4n female parent is homozygous for alleles at each of SNP 34078247, SNP 5640046, SNP 5655752, SNP 5828256, and SNP 5828333.

In various embodiments, 3n watermelon is homozygous for a seed size QTL on chromosome 2 associated with a T allele of SNP 34078247 and/or for a T allele of SNP 34078247. In various embodiments, 3n watermelon is homozygous for a seed size QTL on chromosome 6 associated with an A allele of SNP 5640046 and/or is homozygous for an A allele of SNP 5640046. In various embodiments, 3n watermelon is homozygous for a seed size QTL on chromosome 6 associated with an A allele of SNP 5828256, and/or is homozygous for an A allele of SNP 5828256. In various embodiments, 3n watermelon is homozygous for a seed size QTL on chromosome 6 associated with a C allele of SNP 5828256, and/or is homozygous for a C allele of SNP 5828256. In various embodiments, 3n watermelon is homozygous for a seed size QTL on chromosome 2 associated with a T allele of SNP 34078247 and/or for a T allele of SNP 34078247, is homozygous for a seed size QTL on chromosome 6 associated with an A allele of SNP 5640046 and/or is homozygous for an A allele of SNP 5640046, and further is homozygous for a seed size QTL on chromosome 6 associated with an A or C allele of SNP 5828256, and/or is homozygous for an A or C allele of SNP 5828256.

As will be apparent throughout the disclosure, and parents and crosses described herein and above, can optionally include that one or both parents carry at least one spotted phenotype allele. In various embodiments, at least one parent is heterozygous for a spotted phenotype allele. In various embodiments, both parents are heterozygous for a spotted phenotype allele. In various embodiments, at least one parent is homozygous for a spotted phenotype allele. In various embodiments, both parents are homozygous for a spotted phenotype allele. In various embodiments, one parent is heterozygous for a spotted phenotype allele and the other parent is homozygous for a spotted phenotype allele. In various embodiments, the cross produces a 3n plant genetically and/or phenotypically characterized by a 33% spotted phenotype. In various embodiments, the cross produces a 3n plant genetically and/or phenotypically characterized by a 67% spotted phenotype. In various embodiments, the cross produces a 3n plant genetically and/or phenotypically characterized by a 100% spotted phenotype.

As will be apparent throughout the disclosure, 3n plants described herein and/or above can optionally include at least one spotted phenotype allele. In various embodiments, a 3n plant described herein and/or above can have one spotted phenotype allele. In various embodiments, a 3n plant described herein and/or above can have two spotted phenotype alleles. In various embodiments, a 3n plant described herein and/or above can have three spotted phenotype alleles. In various embodiments, a 3n plant described herein and/or above can be genetically and/or phenotypically characterized by a 33% spotted phenotype. In various embodiments, a 3n plant described herein and/or above can be genetically and/or phenotypically characterized by a 67% spotted phenotype. In various embodiments, a 3n plant described herein and/or above can be genetically and/or phenotypically characterized by a 100% spotted phenotype.

The present disclosure further includes the recognition that in various embodiments genotypes and phenotypes disclosed herein permit those of skill in the art to produce a seed having a given seed size (as determined by the genotype of the female parent) but which, upon fruit production, will yield fruit with seeds of a different seed size. Without wishing to be bound by any particular preferred embodiments, some growers prefer to plant ss4 seeds that produce fruit with ss1, ss2, ss3, ps1, ps2, or ps3 seeds or pips. In some embodiments, the present disclosure provides, among other things, ss4 seeds that produce fruit with ss1, ss2, ss3, ps1, ps2, or ps3 seeds or pips. In some embodiments, the present disclosure provides, among other things, ss4 seeds that produce fruit with ss1, ss2, ps1, or ps2 seeds or pips. In some embodiments, the present disclosure provides, among other things, ss4 seeds that produce fruit with ss2 or ps2 seeds or pips. In some embodiments, the present disclosure provides, among other things, ss4 seeds that produce fruit with ss1 or ps1 seeds or pips.

In some embodiments, the present disclosure provides, among other things, ss3 seeds that produce fruit with ss1, ss2, ps1, or ps2 seeds or pips. In some embodiments, the present disclosure provides, among other things, ss3 seeds that produce fruit with ss2 or ps2 seeds or pips. In some embodiments, the present disclosure provides, among other things, ss3 seeds that produce fruit with ss1 or ps1 seeds or pips.

### Seedless Watermelon Varieties

The present disclosure includes watermelon varieties useful for production of seedless watermelon fruits, including in particular 3n hybrids that consistently produce seedless watermelon fruits. In various embodiments, such seedless watermelon varieties of the present disclosure produce seedless watermelon fruits having traits that are advantageous and/or desirable for production and/or commercialization, including without limitation a seed size in accordance with the present disclosure, advantageously sized pips in accordance with the present disclosure, and/or a spotted phenotype in accordance with the present disclosure. The present disclosure provides, among other things, 3n varieties characterized by spotted genotypes, phenotypes, and/or breeding history or parentage in accordance with the present disclosure. The present disclosure provides, among other things, 3n varieties characterized by seed size genotypes, phenotypes, and/or breeding history or parentage in accordance with the present disclosure. The present disclosure provides, among other things, 3n varieties characterized by both (a) spotted genotypes, phenotypes, and/or breeding history or parentage in accordance with the present disclosure and (b) seed size genotypes, phenotypes, and/or breeding history or parentage in accordance with the present disclosure.

Those of skill in the art will appreciate from the present disclosure that seedless watermelon varieties and fruits thereof in accordance with the present disclosure will demonstrate associated advantageous traits as disclosed herein, and moreover that the achievement of those advantageous is not limited to any particular genetic background. QTLs and SNPs disclosed herein can be identified, selected, and/or used in breeding (including for introgression) across diverse varieties and genetic backgrounds of watermelon. Those of skill in the art will appreciate from the present disclosure that introgression of QTLs and SNPs disclosed herein into diverse varieties and genetic backgrounds of watermelon will cause and/or contribute to expression of associated advantageous phenotypes disclosed herein.

The present disclosure provides, among other things, certain exemplary, non-limiting, watermelon varieties that demonstrate genotypes and phenotypes disclosed herein, including without limitation a triploid watermelon variety designated ‘E26S.00185’, and a triploid watermelon variety designated ‘E26S.00171’.

### Watermelon Variety ‘E26S.00185’

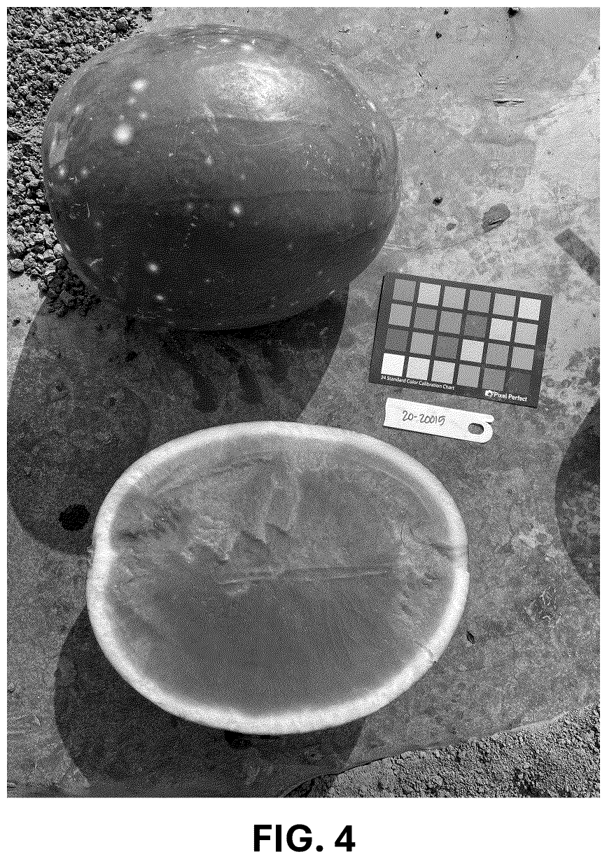

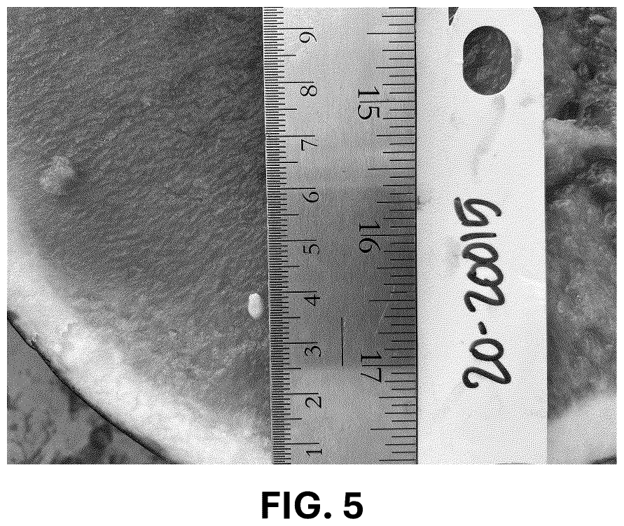

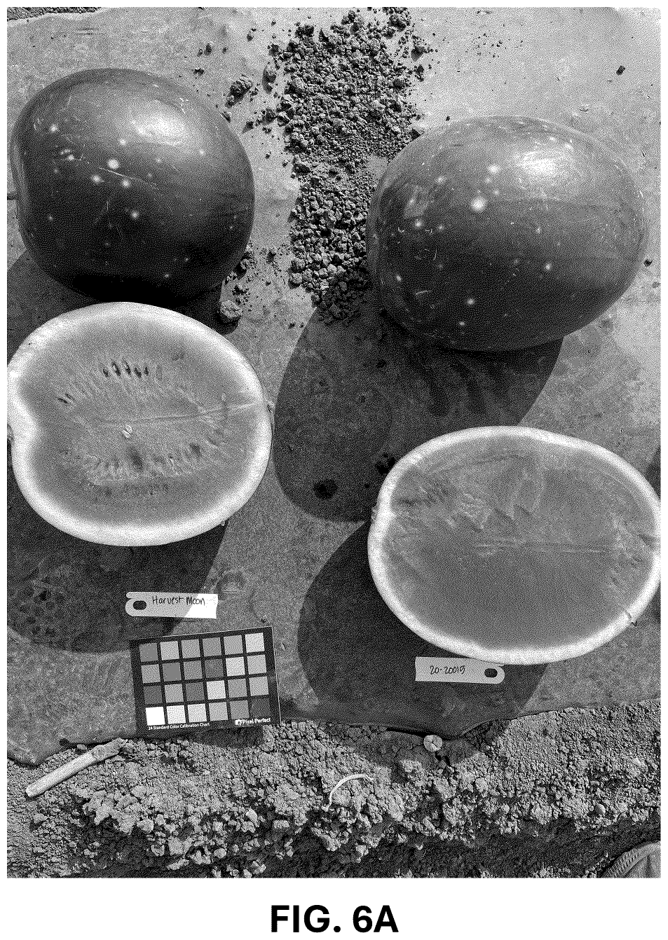

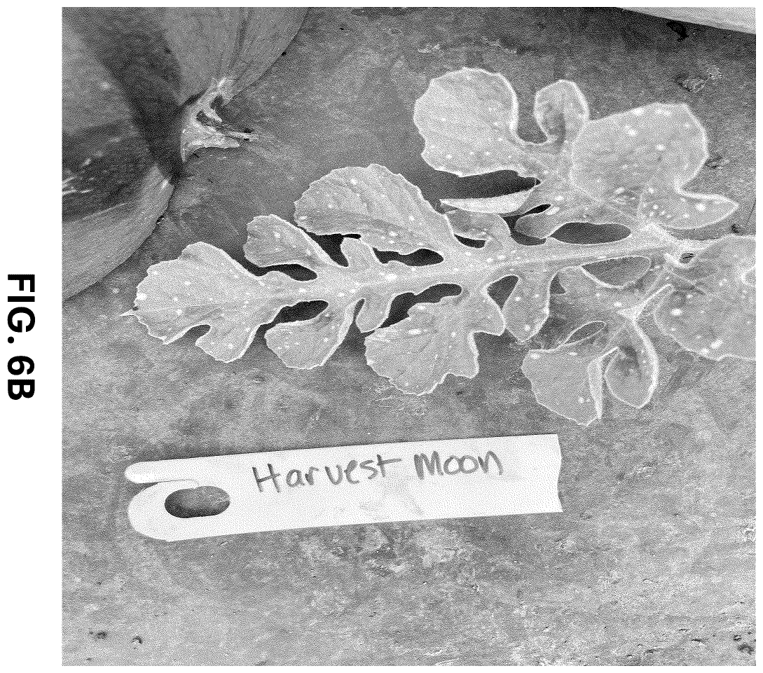

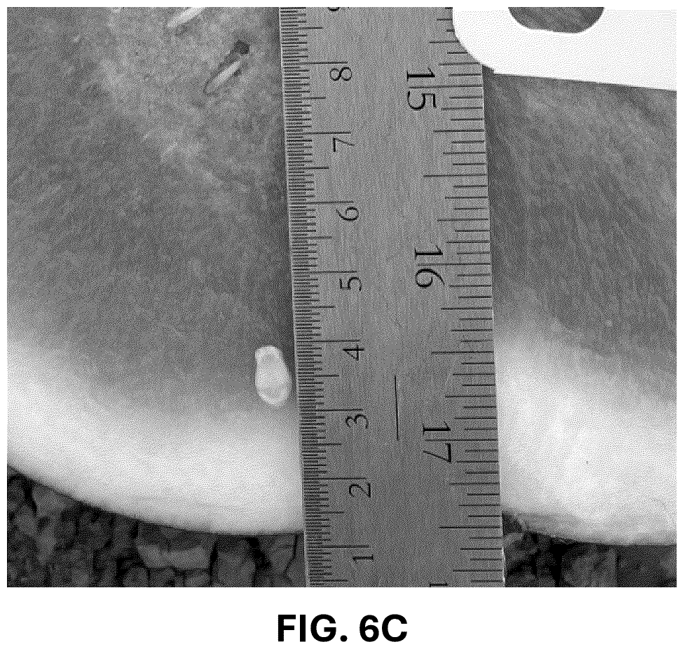

The present disclosure provides, among other things, a triploid watermelon (Citrullus lanatus) variety seed designated as ‘E26S.00185’ and having NCIMB Accession Number 44130. ‘E26S.00185’ is characterized by a 33% spotted phenotype pips of a size advantageous for commercialization. ‘E26S.00185’ is produced by crossing a 4n female parent characterized by a lack of spotted phenotype (0% spotted phenotype) and as having a seed size of (i.e., as having been produced from a seed having a size of) 3 (7.4 g 100 SdW) with a 2n male parent characterized as having a 100% spotted phenotype and a seed size of 2 (1.9 g 100 SdW). Genotypic and phenotypic characteristics of ‘E26S.00185’ and its parental lines are shown in Table 7. ‘E26S.00185’ is further characterized by other advantageous traits, including traits that render the seedless fruit of ‘E26S.00185’ desirable for consumption and commercialization. FIG. 4 shows whole and cross-sectioned fruit of watermelon variety ‘E26S.00185’. FIG. 5 shows the size of pips of seedless fruit of watermelon variety ‘E26S.00185’. FIG. 6A shows the spotted rind phenotype of seedless watermelon of Harvest Moon (left) and watermelon variety ‘E26S.00185’ (right). FIG. 6B shows the spotted leaf phenotype of Harvest Moon. FIG. 6C shows the size of pips of seedless fruit of Harvest Moon.

TABLE 7
‘E26S.00185’ and parent genotype and phenotype information

| | | Seed | | SNP spotted | | SNP | | | | |
|---|---|---|---|---|---|---|---|---|---|---|
| | % | size | | phenotype | | seed size | | | | |
| | Spotted | (ss1; 2, 3, 4) | 100sdw | 4:134886 | 4:86340 | 2:34078247 | 6:5640046 | 6:5655752 | 6:5828256 | 6:5828333 |
| 4n | 0 | ss3 | 7.4 | AAAA | AAAA | TTTT | AAAA | TTTT | CCCC | AAAA |
| 2n | 100 | ss2 | 1.9 | TT | GG | TT | AA | TT | AA | GG |
| 3n | 33 | ss3 | 6.5 | TAA | GAA | TTT | AAA | TTT | CCA | GAA |

In one embodiment, the present disclosure provides a Citrullus lanatus watermelon plant produced by growing ‘E26S.00185’ watermelon seed and/or parts isolated therefrom (which plants and parts can be referred to, e.g., as ‘E26S.00185’ plants and ‘E26S.00185’ parts, respectively). In some embodiments, the present disclosure provides a Citrullus lanatus seed, plants grown from the seed, having all, or essentially all, the physiological and morphological characteristics of a Citrullus lanatus plant produced by growing ‘E26S.00185’ watermelon seed having NCIMB Accession Number 44130. and/or parts isolated therefrom.

Watermelon plant parts include watermelon leaves, ovules, pollen, seeds, watermelon fruits, parts of watermelon fruits, flowers, cells, and/or the like. In some embodiments, the present disclosure provides watermelon leaves, ovules, pollen, seeds, watermelon fruits, parts of watermelon fruits, and/or flowers isolated from ‘E26S.00185’ watermelon plants. In some embodiments, the present disclosure provides pollen grains or ovules isolated from ‘E26S.00185’ watermelon plants. In some embodiments, the present disclosure provides protoplasts produced from ‘E26S.00185’ watermelon plants. In some embodiments, the present disclosure provides tissue or tissue culture produced from protoplasts or cells of ‘E26S.00185’ watermelon plants, wherein said cells or protoplasts are produced from a plant part selected from the group of root, root tip, meristematic cell, stem, hypocotyl, petiole, cotyledon, leaf, flower, anther, pollen, pistil, and/or fruit. The present disclosure provides watermelon plants regenerated from the tissue culture, where the plants regenerated from the tissue culture have all, or essentially all, of the morphological and physiological characteristics of ‘E26S.00185’ watermelon.

In some embodiments, the present disclosure provides a method of producing seedless watermelon fruit, the method including: (a) crossing a first ‘E26S.00185’ watermelon plant, representative sample of seed having been deposited under NCIMB Accession Number 44130, with a second diploid watermelon plant; (b) allowing seedless fruit to form; and (c) harvesting the seedless fruit.

In some embodiments, the present disclosure provides methods of vegetatively propagating a plant of watermelon variety ‘E26S.00185’, the method including the steps of: (a) collecting tissue capable of being propagated from a plant of watermelon variety ‘E26S.00185’, representative seed of said watermelon variety ‘E26S.00185’ having been deposited under NCIMB Accession Number 44130; and (b) producing a rooted plant from said tissue.

According to the disclosure, there is provided a watermelon plant designated ‘E26S.00185’. This disclosure thus relates to the plants of watermelon ‘E26S.00185’, the plant parts of watermelon ‘E26S.00185’, and to the seeds of watermelon ‘E26S.00185’. This disclosure also relates to methods for producing other watermelon cultivars or hybrids derived from watermelon ‘E26S.00185’ and to the watermelon cultivars and hybrids derived by the use of those methods.

In some embodiments, the present disclosure provides single gene converted plants of watermelon ‘E26S.00185’. The single transferred gene may preferably be a dominant or recessive allele. In various embodiments, the single transferred gene can confer a trait such as sex determination, herbicide resistance, insect resistance, resistance for bacterial, fungal, or viral disease, improved harvest characteristics, enhanced nutritional quality, and/or improved agronomic quality. The single gene may be a naturally occurring watermelon gene or a transgene introduced through genetic engineering techniques.

In some embodiments, the present disclosure provides methods for developing watermelon plants in a watermelon plant breeding program using plant breeding techniques including recurrent selection, backcrossing, pedigree breeding, restriction fragment length polymorphism enhanced selection, genetic marker enhanced selection, and/or transformation. Marker loci such as restriction fragment polymorphisms or random amplified DNA have been published for many years and may be used for selection (See, Pierce et al., HortScience (1990) 25:605-615; Wehner, T., Cucurbit Genetics Cooperative Report, (1997) 20: 66-88; and Kennard et al., Theoretical Applied Genetics (1994) 89:217-224). Seeds, watermelon plants, and parts thereof produced by such breeding methods are also part of the disclosure.

In some embodiments, the present disclosure provides watermelon seeds resulting from methods of making a watermelon variety of the present disclosure. In additional embodiments, the present disclosure provides watermelon plants, and/or parts thereof, obtained from growing the seeds of the present disclosure. In additional embodiments, the present disclosure provides watermelon plants, and/or parts thereof, having all, or essentially all, the physiological and morphological characteristics of the watermelon plants of the present disclosure. In additional embodiments, the present disclosure provides watermelon tissue culture, obtained from the plants of the present disclosure. In some embodiments, the tissue culture of the present disclosure is produced from a plant part selected from the group consisting of leaf, anther, pistil, stem, petiole, root, root tip, fruit, seed, flower, cotyledon, hypocotyl, embryo, and/or meristematic cell.

In addition to the exemplary aspects and embodiments described above, further aspects and embodiments will become apparent by reference by study of the following descriptions.

A deposit of at least 625 seeds of the watermelon variety ‘E26S.00185’ was made with the National Collection of Industrial, Food and Marine Bacteria Ltd. (NCIMB Ltd), Wellheads Place, Dyce, Aberdeen, AB21 7 GB, United Kingdom, and assigned NCIMB Number 44130. The seeds deposited with the NCIMB on [DATE] were obtained from the seed of the variety maintained by Enza Zaden USA, Inc., 7 Harris Place, Salinas, California 93901, United States since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the watermelon variety ‘E26S.00185’ will be maintained in the NCIMB, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Watermelon Variety ‘E26S.00171’

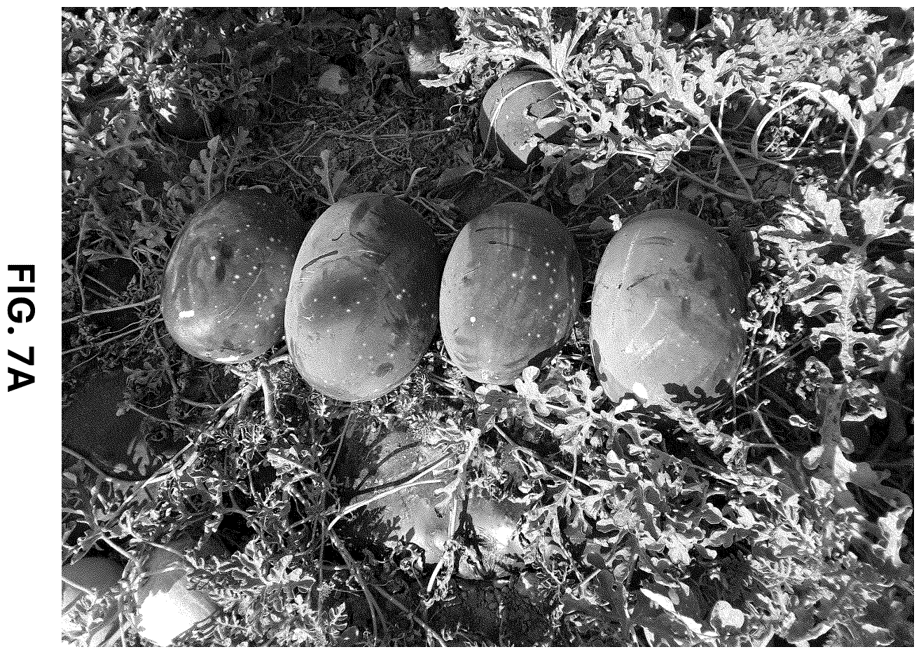

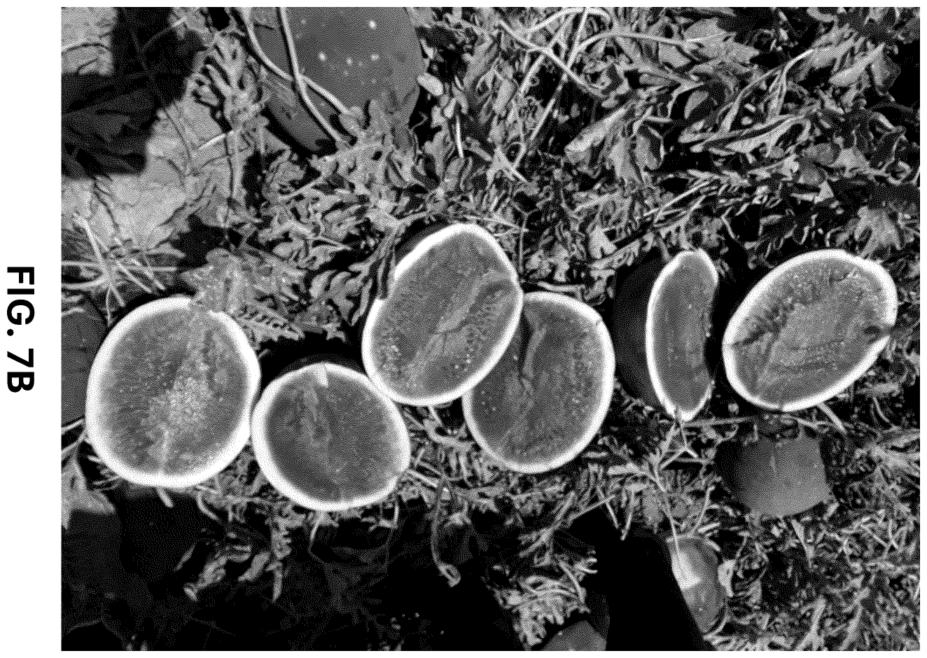

The present disclosure provides, among other things, a triploid watermelon (Citrullus lanatus) variety seed designated as ‘E26S.00171’ and having NCIMB Accession Number X2. ‘E26S.00171’ is characterized by a 33% spotted phenotype and ps3 pips. ‘E26S.00171’ is produced by crossing a 4n female parent characterized by a lack of spotted phenotype (0% spotted phenotype) and as having a seed size of (i.e., as having been produced from a seed having a size of) 3 (7.2 g 100 SdW) with a 2n male parent characterized as having a 100% spotted phenotype and a seed size of 5 (9.6 g 100 SdW). Genotypic and phenotypic characteristics of ‘E26S.00171’ and its parental lines are shown in Table 8. Seeds for production of ‘E26S.00171’ watermelon plants can have a weight of 2-5 100 SdW. ‘E26S.00171’ is further characterized by other advantageous traits, including traits that render the seedless fruit of ‘E26S.00171’ desirable for consumption and commercialization. FIG. 7A shows whole ‘E26S.00171’ fruits at maturity. FIG. 7B shows longitudinal cross-sections of ‘E26S.00171’ fruit.

TABLE 8
‘E26S.00171’ and parent genotype and phenotype information

| | | | | SNP spotted | | SNP | | | | |
|---|---|---|---|---|---|---|---|---|---|---|
| | % | Seed | | phenotype | | seed size | | | | |
| | Spotted | size | 100sdw | 4:134886 | 4:86340 | 2:34078247 | 6:5640046 | 6:5655752 | 6:5828256 | 6:5828333 |
| 4n | 0 | ss3 | 7.2 | AAAA | AAAA | TTTT | AAAA | TTTT | CCCC | AAAA |
| 2n | 100 | ss4 | 9.6 | TT | GG | TTT | CC | CC | CC | AA |
| 3n | 33 | ss3 | 6.3 | TAA | GAA | TTT | CAA | CTT | CCC | AAA |

In one embodiment, the present disclosure provides a Citrullus lanatus watermelon plant produced by growing ‘E26S.00171’ watermelon seed and/or parts isolated therefrom (which plants and parts can be referred to, e.g., as ‘E26S.00171‘ plants and’E26S.00171’ parts, respectively). In some embodiments, the present disclosure provides a Citrullus lanatus seed, plants grown from the seed, having all, or essentially all, the physiological and morphological characteristics of a Citrullus lanatus plant produced by growing ‘E26S.00171’ watermelon seed having NCIMB Accession Number X2. and/or parts isolated therefrom.

Watermelon plant parts include watermelon leaves, ovules, pollen, seeds, watermelon fruits, parts of watermelon fruits, flowers, cells, and/or the like. In some embodiments, the present disclosure provides watermelon leaves, ovules, pollen, seeds, watermelon fruits, parts of watermelon fruits, and/or flowers isolated from ‘E26S.00171’ watermelon plants. In some embodiments, the present disclosure provides pollen grains or ovules isolated from ‘E26S.00171’ watermelon plants. In some embodiments, the present disclosure provides protoplasts produced from ‘E26S.00171’ watermelon plants. In some embodiments, the present disclosure provides tissue or tissue culture produced from protoplasts or cells of ‘E26S.00171’ watermelon plants, wherein said cells or protoplasts are produced from a plant part selected from the group of root, root tip, meristematic cell, stem, hypocotyl, petiole, cotyledon, leaf, flower, anther, pollen, pistil, and/or fruit. The present disclosure provides watermelon plants regenerated from the tissue culture, where the plants regenerated from the tissue culture have all, or essentially all, of the morphological and physiological characteristics of ‘E26S.00171’ watermelon.

In some embodiments, the present disclosure provides a method of producing seedless watermelon fruit, the method including: (a) crossing a first ‘E26S.00171’ watermelon plant, representative sample of seed having been deposited under NCIMB Accession Number X2, with a second diploid watermelon plant; (b) allowing seedless fruit to form; and (c) harvesting the seedless fruit.

In some embodiments, the present disclosure provides methods of vegetatively propagating a plant of watermelon variety ‘E26S.00171’, the method including the steps of: (a) collecting tissue capable of being propagated from a plant of watermelon variety ‘E26S.00171’, representative seed of said watermelon variety ‘E26S.00171’ having been deposited under NCIMB Accession Number X2; and (b) producing a rooted plant from said tissue.

According to the disclosure, there is provided a watermelon plant designated ‘E26S.00171’. This disclosure thus relates to the plants of watermelon ‘E26S.00171’, the plant parts of watermelon ‘E26S.00171’, and to the seeds of watermelon ‘E26S.00171’. This disclosure also relates to methods for producing other watermelon cultivars or hybrids derived from watermelon ‘E26S.00171’ and to the watermelon cultivars and hybrids derived by the use of those methods.

In some embodiments, the present disclosure provides single gene converted plants of watermelon ‘E26S.00171’. The single transferred gene may preferably be a dominant or recessive allele. In various embodiments, the single transferred gene will confer such trait as sex determination, herbicide resistance, insect resistance, resistance for bacterial, fungal, or viral disease, improved harvest characteristics, enhanced nutritional quality, and/or improved agronomic quality. The single gene may be a naturally occurring watermelon gene or a transgene introduced through genetic engineering techniques.

In some embodiments, the present disclosure provides methods for developing watermelon plants in a watermelon plant breeding program using plant breeding techniques including recurrent selection, backcrossing, pedigree breeding, restriction fragment length polymorphism enhanced selection, genetic marker enhanced selection, and/or transformation. Marker loci such as restriction fragment polymorphisms or random amplified DNA have been published for many years and may be used for selection (See, Pierce et al., HortScience (1990) 25:605-615; Wehner, T., Cucurbit Genetics Cooperative Report, (1997) 20: 66-88; and Kennard et al., Theoretical Applied Genetics (1994) 89:217-224). Seeds, watermelon plants, and parts thereof produced by such breeding methods are also part of the disclosure.

In some embodiments, the present disclosure provides watermelon seeds resulting from methods of making a watermelon variety of the present disclosure. In additional embodiments, the present disclosure provides watermelon plants, and/or parts thereof, obtained from growing the seeds of the present disclosure. In additional embodiments, the present disclosure provides watermelon plants, and/or parts thereof, having all, or essentially all, the physiological and morphological characteristics of the watermelon plants of the present disclosure. In additional embodiments, the present disclosure provides watermelon tissue culture, obtained from the plants of the present disclosure. In some embodiments, the tissue culture of the present disclosure is produced from a plant part selected from the group consisting of leaf, anther, pistil, stem, petiole, root, root tip, fruit, seed, flower, cotyledon, hypocotyl, embryo, and/or meristematic cell.

In addition to the exemplary aspects and embodiments described above, further aspects and embodiments will become apparent by reference by study of the following descriptions.

A deposit of at least 625 seeds of the watermelon variety ‘E26S.00171’ was made with the National Collection of Industrial, Food and Marine Bacteria Ltd. (NCIMB Ltd), Wellheads Place, Dyce, Aberdeen, AB21 7 GB, United Kingdom, and assigned NCIMB Number X2. The seeds deposited with the NCIMB on [DATE] were obtained from the seed of the variety maintained by Enza Zaden USA, Inc., 7 Harris Place, Salinas, California 93901, United States since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the watermelon variety ‘E26S.00171’ will be maintained in the NCIMB, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Pollinizer Watermelon for Seedless Fruit Production

A pollinizer is a 2n watermelon that is intermixed in fields with 3n plants, and which is used as a source of pollen to promote 3n watermelon fruit set. In various embodiments, pollinizer plants can also produce fruit by self-pollination. However, pollinizer fruit is typically not the desired production product (rather, the primary consumer product is the triploid seedless watermelon fruit). In some instances, pollinizer fruits are discarded, while in others they may be edible and channeled toward a particular or alternative use that is distinct from the use of the seedless 3n watermelon fruits. Because the pollinizer fruits and seedless 3n watermelon plants are intermixed in watermelon fields, pollinizer plants that can be visually distinguished from seedless 3n watermelon plants are advantageous for production, allowing separation during harvesting. To provide just one example of a pollinizer trait, in some embodiments a pollinizer produces fruits that are smaller than 3n fruits and thus can be readily distinguished. The present disclosure provides pollinizers that are distinguished by a spotted pattern, e.g., where 3n seedless watermelon plants, plant parts (e.g., leaves), and fruits have a different spotted pattern or no spotted pattern.

The present disclosure further recognizes that the pollinizer plants are intermixed with 3n fruit-producing hybrids in watermelon planting schemes and process, and in fields, pollinizer plants that can be visually distinguished from seedless 3n watermelon plants (e.g., prior to fruiting) are further advantageous because such pollinizers allow efficient recognition of pollinizer plants during planting and growth. Pollinizers that are distinguishable from 3n plants by a spotted phenotype of the present disclosure provide this advantage as well, at least because plants and plant parts (e.g., leaves) of plants that have a spotted phenotype display the spotting.

In various embodiments, the present disclosure provides a 2n pollinizer watermelon that is homozygous or heterozygous for spotted alleles, for pollination of 3n plants that do not include a spotted allele. Accordingly, while 3n seedless watermelon are not spotted, pollinizer fruits are 100% spotted or 50% spotted. In various such embodiments, a pollinizer watermelon is homozygous for a T allele of SNP 134886 and/or homozygous for a G allele of SNP 86340, and a 3n watermelon is homozygous for an A allele of SNP 134886 and homozygous for an A allele of SNP 86340. In various such embodiments, a pollinizer watermelon is heterozygous for a T allele of SNP 134886 and/or heterozygous for a G allele of SNP 86340, and a 3n watermelon is homozygous for an A allele of SNP 134886 and homozygous for an A allele of SNP 86340.

In various embodiments, the present disclosure provides a 2n pollinizer watermelon that does not include spotted alleles, for pollination of 3n plants that include at least one spotted allele. Accordingly, 3n seedless watermelon are spotted (e.g., 33%, 67%, or 100% spotted), while pollinizer fruits are not spotted. In various such embodiments, a pollinizer watermelon is homozygous for an A allele of SNP 134886 and homozygous for an A allele of SNP 86340, and a 3n watermelon includes at least one T allele of SNP 134886 and at least one G allele of SNP 86340 (e.g., one T allele of SNP 134886 and one G allele of SNP 86340; two T alleles of SNP 134886 and two G alleles of SNP 86340; or three T alleles of SNP 134886 and three G alleles of SNP 86340, e.g., where each T allele of SNP 134886 is present on the same chromosome as a G allele of SNP 86340).

In various embodiments, the present disclosure provides a 2n pollinizer watermelon that is homozygous or heterozygous for spotted alleles, for pollination of 3n plants that include one spotted allele or two spotted alleles. Accordingly, 3n seedless watermelon are spotted (e.g., 33% or 67%, spotted), while pollinizer fruits are 50% or 100% spotted. In various such embodiments, a pollinizer watermelon is homozygous a T allele of SNP 134886 and/or homozygous for a G allele of SNP 86340, and a 3n watermelon includes either one T allele of SNP 134886 and one G allele of SNP 86340, or two T alleles of SNP 134886 and two G alleles of SNP 86340. In various such embodiments, a pollinizer watermelon is heterozygous for a T allele of SNP 134886 and/or heterozygous for a G allele of SNP 86340, and a 3n watermelon includes either one T allele of SNP 134886 and one G allele of SNP 86340, or two T alleles of SNP 134886 and two G alleles of SNP 86340.

In various embodiments, a pollinizer of the present disclosure can have any spotted genotype and/or spotted phenotype provided herein, and can further have any of a variety of other pollinizer traits known to those of skill in the art. Other pollinizer traits know to those of skill in the art include, without limitation, compact growth habit, high pollen production, timing of flowering (e.g., synchronized with 3n plant flowering), and/or small fruit size.

In various embodiments, a 3n watermelon in combination with pollinizer watermelon provided herein can have traits set forth in conjunction with the selection of pollinizer (e.g., with respect to spot phenotype) and any further genotypic and/or phenotypic traits provided herein, including without limitation any seed size genotype and/or phenotype provided herein.

### Seeded Watermelon Varieties

The present disclosure further recognizes and encompasses embodiments in which seeded watermelon are produced, e.g., for human consumption. Seeded watermelon production is a further aspect in which seed size QTLs and SNPs of the present disclosure are useful in breeding and production of plants for commercial sale. The present disclosure includes the recognition that consumers of seeded watermelon generally prefer small or medium (ss1, ss2, or ss3) seeds in seeded watermelon fruits. In certain embodiments, seeded watermelon having ss1 or ss2 seed size, e.g., seeded watermelon having ss1 seed size, can be particularly advantageous for commercial use. Seeded watermelon products are typically 2n, and can have genotypes and phenotypes as set forth in the following table:

TABLE 9
Exemplary 2n Genotypes for Seeded Watermelon

| | Genotype | | | Seed Size in |
|---|---|---|---|---|
| | 2:34078247 | 6:5640046 | 6:5828256 | Fruit Products |
| 1 | AA | CC | CC | ss1 |
| | AA | CC | CA | ss1 |
| | AA | CC | AA | ss1 |
| | AA | CA | CC | ss1 |
| | AA | CA | CA | ss1 |
| | AA | CA | AA | ss1 |
| | AA | AA | CC | ss1 |
| | AA | AA | CA | ss1 |
| | AA | AA | AA | ss1 |
| | TT/AT | CC | CC | ss4 |
| | TT/AT | CC | CA | ss3 |
| | TT/AT | CC | AA | ss3 |
| | TT/AT | CA | CC | ss3 |
| | TT/AT | CA | CA | ss2 |
| | TT/AT | CA | AA | ss2 |
| | TT/AT | AA | CC | ss3 |
| | TT/AT | AA | CA | ss2 |
| | TT/AT | AA | AA | ss2 |

As will be apparent throughout the disclosure, 2n plants described herein and/or above can optionally include at least one spotted phenotype allele. In various embodiments, a 2n plant described herein and/or above can have one spotted phenotype allele. In various embodiments, a 2n plant described herein and/or above can have two spotted phenotype alleles. In various embodiments, a 3n plant described herein and/or above can be genetically and/or phenotypically characterized by a 50% spotted phenotype. In various embodiments, a 3n plant described herein and/or above can be genetically and/or phenotypically characterized by a 100% spotted phenotype.

The present disclosure further includes the recognition that in various embodiments genotypes and phenotypes disclosed herein permit those of skill in the art to produce a seed having a given seed size (as determined by the genotype of the female parent) but which, upon fruit production, will yield fruit with seeds of a different seed size. Without wishing to be bound by any particular preferred embodiments, some growers prefer to plant ss4 seeds that produce fruit with ss1, ss2, or ss3 seeds. In some embodiments, the present disclosure provides, among other things, ss4 seeds that produce fruit with ss1, ss2 or ss3 seeds. In some embodiments, the present disclosure provides, among other things, ss4 seeds that produce fruit with ss1 or ss2 seeds. In some embodiments, the present disclosure provides, among other things, ss4 seeds that produce fruit with ss2 seeds. In some embodiments, the present disclosure provides, among other things, ss4 seeds that produce fruit with ss1 seeds.

In some embodiments, the present disclosure provides, among other things, ss3 seeds that produce fruit with ss1 or ss2 seeds. In some embodiments, the present disclosure provides, among other things, ss3 seeds that produce fruit with ss2 seeds. In some embodiments, the present disclosure provides, among other things, ss3 seeds that produce fruit with ss1 seeds.

### EXAMPLES

The present Examples demonstrate SNP alleles associated with a seed size and SNP alleles associated with a spotted phenotype. The present Examples therefore demonstrate to those of skill in the art that methods and compositions of the present disclosure are useful to produce watermelon with advantageous seed size as disclosed herein, as well as watermelon having a spotted phenotype.

### Example 1: SNP Alleles and QTLs Associated with Seed Size

The present disclosure is based at least in part on the detailed genetic analysis of SNPs and QTLs that are associated with seed size.

The present disclosure includes the identification of a QTL on chromosome 2 that is associated with a very small seed size phenotype referred to as “ss1” seed size. In a SNP at position 34078247 associated with this QTL can have an A allele or a T allele. In 2n watermelon, plants homozygous for the A allele of SNP 34078247 have a ss1 seed size, whereas plants with any other genotype (e.g., heterozygous, or homozygous for the T allele of SNP 34078247) do not have a ss1 seed size and do not appear to limit seed size.

The present disclosure includes the identification of a QTL on chromosome 6 that is associated with a large size phenotype referred to as “ss4” seeds. In a SNP at position 5640046 associated with this QTL can have a C allele or an A allele. In 2n watermelon, plants homozygous for the C allele of SNP 5640046 can have ss4 seeds, whereas plants with any other genotype (e.g., heterozygous, or homozygous for the A allele of SNP 5640046) do not have ss4 seeds and do not appear to cause or contribute to large seed size. A like-performing SNP was also identified at position 5655752, which can have a C allele or a T allele. SNP 5655752 was found to be a comparable alternative to SNP 5640046. In 2n watermelon, plants homozygous for the C allele of SNP 5655752 can have ss4 seeds, whereas plants with any other genotype (e.g., heterozygous, or homozygous for the T allele of SNP 5655752) do not have ss4 seeds and do not appear to cause or contribute to large seed size.

The present disclosure includes the identification of a further QTL on chromosome 6 that is associated with a large size phenotype, “ss3” to “ss4” seeds. In a SNP at position 5828256 associated with this QTL can have an C allele or an A allele. In 2n watermelon, plants homozygous for the C allele of SNP 5828256 can have ss3-5 seeds, whereas plants with any other genotype (e.g., heterozygous, or homozygous for the A allele of SNP 5828256) do not have ss4 seeds and do not appear to cause or contribute to large seed size. A like-performing SNP was also identified at position 5828333, which can have an A allele or a G allele. SNP 5828333 was found to be a comparable alternative to SNP 5828256. In 2n watermelon, plants homozygous for the A allele of SNP 5828333 can have ss3-5 seeds, whereas plants with any other genotype (e.g., heterozygous, or homozygous for the G allele of SNP 5828333) do not have ss4 seeds and do not appear to cause or contribute to large seed size.

It is further appreciated that a large seed results if a 2n plant is homozygous for (a) one or both of the C allele of SNP and 5640046 the C allele of SNP 5655752, and (b) one or both of the C allele of SNP 5828256 A allele of SNP 5828333. All other combinations of alleles produces ss2 or ss3 seeds that are advantageous for production and fruit quality.

TABLE 10
SNP Alleles and QTLs Associated with Seed Size

| | | Position and | | |
|---|---|---|---|---|
| QTL | Chromosome | alternative(s) | SNP | Characterization (2n) |
| 1 | 2 | 34078247 | T/A | AA causes ssl seed size |
| | | | | AT, TT do not limit seed |
| 2 | 6 | 5640046 | C/A | CC contributes to ss3 or ss4 |
| | | | | CA, AA do not cause ss3 or ss4 |
| | | 5655752 | C/T | CC contributes to ss3 or ss4 |
| | | | | CT, TT do not cause ss3 or ss4 |
| 3 | 6 | 5828256 | C/A | CC contributes to seed ss3 or ss4 |
| | | | | CA, AA do not cause ss3 or ss4 |
| | | 5828333 | A/G | AA contributes to seed ss3 or ss4 |
| | | | | AG, GG do not cause ss3 or ss4 |

TABLE 11
Exemplary genotypes and phenotypes

| | Seed Size Marker Allele(s) | | | Seed | 100SdW |
|---|---|---|---|---|---|
| Genotype(s) | 2:34078247 | 6:5640046 | 6:5828256 | Size | (g) |
| 1 | AA | AA | AA | ss1 | 0.2-1.1 |
| | | CC | CC | | |
| | | AC | AC | | |
| 2 | TT | AA | AA | ss2 | 1.1-3.2 |
| | AT | AC | AC | | |
| 3 | TT | AA | CC | ss3 | 2.7-5.9 |
| | AT | AC | | | |
| 4 | TT | CC | AA | ss3 | |
| | AT | | | | |
| 5 | TT | CC | CC | ss4 | 4.9-1.7 |
| | AT | | | | |

Certain SNP alleles of the present Example were detected using the following exemplary primers and probes based on the Charleston Gray reference genome:

TABLE 12
Exemplary primers and probes

| | | | SEQ |
|---|---|---|---|
| | Primer/ | | ID |
| Marker | Probe | Sequence | NO: |
| Chromosome | Forward | ATTGCCCCAAGCACAGAGATA | 9 |
| 2, SNP | Primer | | |
| 34078247: | Reverse | AAATTGGTATAACTGTGGCAAGAA | 10 |
| | Primer | | |
| | Probe | TAGATCTCTCAACACACtTTGCAATGCAAt | 11 |
| Chromosome | Forward | TTTGAGTGGTTACCGAACATTCT | 12 |
| 6, SNP | Primer | | |
| 5640046 | Reverse | TCTTTGTATGCTCACGAATTTCA | 13 |
| | Primer | | |
| | Probe | TTGTTAAACTAATcGGTGAAATTTCAAAGc | 14 |
| Chromosome | Forward | TTTGCTTTTTCAAATTACCACTAA | 15 |
| 6, SNP | Primer | | |
| 5655752 | Reverse | TTTTTCTTTTGGAAGCCGAA | 16 |
| | Primer | | |
| | Probe | AAAAACcTCCCCTTCTGATTTGATCTg | 17 |
| Chromosome | Forward | TTATCAGGATTATTTGCACTGAAG | 18 |
| 6, SNP | Primer | | |
| 5828256 | Reverse | ATAGCTTTCACAAGTTTTGGCTCT | 19 |
| | Primer | | |
| | Probe | TTAAACCATATGCTTCCTCcATCACAAAAg | 20 |
| Chromosome | Forward | TTTAGAGCCAAAACTTGTGAAAGC | 21 |
| 6, SNP | Primer | | |
| 5828333 | Reverse | TTATTGCTTTTTGCCCTAATTACA | 22 |
| | Primer | | |
| | Probe | ATTCTGCTAaTTATGTTCGGAAATTGTTGc | 23 |

Those of skill in the art will appreciate that other primers and probes can be selected, designed, and/or utilized in accordance with the sequence context of the provided SNPs based on the Charleston Gray reference genome, which is set forth in non-limiting part below:

TABLE 13
Reference Genomic Sequences

| | | SEQ ID |
|---|---|---|
| Marker | Sequence | NO: |
| Chromosome 2, SNP | CACTAGCTGCGTCATTGCCCCAAGCACAGAGATA | 24 |
| 34078247: | ATTTGAGTTTAGATCTCTCAACACAC[T/A]TTGCAA | |
| | TGCAAAACTTGAGCCTTTTGTTTTCTTGCCACAGT | |
| | TATACCAATTTAGTTGGAA | |
| Chromosome 6, SNP | TGGTTACCGAACATTCTAATTTCTTCTAAAATGAC | 25 |
| 5640046 | TTATTTTTTAAATTAAGCATTTAAAAATATATTCC | |
| | AAACAAATCCTAAATTTTTGTTAAACTAAT[C/A]G | |
| | GTGAAATTTCAAAGGTTATTTATTTATAGGTAACT | |
| | TAAATTTGAAGAGAACATGATATTATTGAAATTCG | |
| | TGAGCATACAAAGATTATTACATTGTTTT | |
| Chromosome 6, SNP | TTTTGCTTTTTCAAATTACCACTAAAAAAAAAAAA | 26 |
| 5655752 | AGAAAAAAAGAAAAAAAGAAAAAAC[C/T]TCCCC | |
| | TTCTGATTTGATCTCTTCGGCTTCCAAAAGAAAAA | |
| | ATGTCCACCAAATCCCCTGT | |
| Chromosome 6, SNP | ATTAGTACTTTAGAATAAAAAAGACAGAAAGACC | 27 |
| 5828256 | AAGAGGATACACTTCAGTTATCAGGATTATTTGCA | |
| | CTGAAGTGTTTATTAAACCATATGCTTCCTC[A/C]A | |
| | TCACAAAACATTTGTGTCATTATTTAGAGCCAAAA | |
| | CTTGTGAAAGCTATACATCATGAGTGTTTTCATTC | |
| | TGCTAATTATGTTCGGAAATTGTTGGAGT | |
| Chromosome 6, SNP | GTCATTATTTAGAGCCAAAACTTGTGAAAGCTATA | 28 |
| 5828333 | CATCATGAGTGTTTTCATTCTGCTA[A/G]TTATGTT | |
| | CGGAAATTGTTGGAGTGTAATTAGGGCAAAAAGC | |
| | AATAAACTCAAAAGGAGAA | |

The present disclosure further includes the recognition that genotypes (e.g., QTLs and SNPs) provided herein with respect to spotted watermelon and/or seed size are significant across diverse watermelon genetic background. Examples of diverse watermelon varieties, their genotypes for relevant seed size QTLs and/or markers thereof, and their seed sizes are provided in Table 14.

TABLE 14
Exemplary 2n and 4n plant seed size genotypes and phenotypes

| | | Seed Size SNP Alleles | | | 100 | Seed |
|---|---|---|---|---|---|---|
| Variety | Ploidy | 2:34078247 | 6:5828256 | 6:5640046 | SdW (g) | Size |
| 1 | 4n | TTTT | CCCC | AAAA | 4.7 | ss3 |
| 2 | 4n | TTTT | CCCC | AAAA | 7.2 | ss3 |
| 3 | 4n | TTTT | CCCC | AAAA | 6 | ss3 |
| 4 | 4n | TTTT | CCCC | CCCC | 10.1 | ss4 |
| 5 | 4n | TTTT | AAAA | AAAA | 5.1 | ss2 |
| 6 | 4n | TTTT | AAAA | AAAA | 3.1 | ss2 |
| 7 | 4n | TTTT | AAAA | AAAA | 7.3 | ss2 |
| 8 | 4n | TTTT | CCCC | AAAA | 5.3 | ss3 |
| 9 | 4n | TTTT | CCCC | AAAA | 7.7 | ss3 |
| 10 | 4n | TTTT | AAAA | AAAA | 7.6 | ss2 |
| 11 | 4n | TTTT | CCCC | AAAA | 1.2 | ss3 |
| 12 | 4n | TTTT | CCCC | AAAA | 6.4 | ss3 |
| 13 | 4n | TTTT | CCCC | AAAA | 5.9 | ss3 |
| 14 | 4n | TTTT | CCCC | AAAA | 2.6 | ss3 |
| 15 | 4n | TTTT | CCCC | AAAA | 5.7 | ss3 |
| 16 | 4n | ND | CCCC | AAAA | 4.9 | ss3 |
| 17 | 4n | TTTT | CCCC | AAAA | 7 | ss3 |
| 18 | 4n | TTTT | CCCC | AAAA | 6.5 | ss3 |
| 19 | 4n | TTTT | CCCC | AAAA | 7.6 | ss3 |
| 20 | 4n | TTTT | CCCC | AAAA | 7.3 | ss3 |
| 21 | 4n | TTTT | CCCC | AAAA | 7 | ss3 |
| 22 | 4n | TTTT | CCCC | AAAA | 4.6 | ss3 |
| 23 | 4n | TTTT | CCCC | AAAA | 6 | ss3 |
| 24 | 4n | TTTT | CCCC | AAAA | 6.9 | ss3 |
| 25 | 4n | TTTT | CCCC | AAAA | 6 | ss3 |
| 26 | 4n | TTTT | CCCC | AAAA | 5.8 | ss3 |
| 27 | 4n | TTTT | CCCC | AAAA | 6.2 | ss3 |
| 28 | 4n | TTTT | CCCC | AAAA | 6.1 | ss3 |
| 29 | 4n | ND | CCCC | AAAA | 7.1 | ss3 |
| 30 | 4n | TTTT | CCCC | AAAA | 6.8 | ss3 |
| 31 | 4n | TTTT | CCCC | AAAA | 7.5 | ss3 |
| 32 | 4n | TTTT | CCCC | AAAA | 7.6 | ss3 |
| 33 | 4n | TTTT | CCCC | AAAA | 7.2 | ss3 |
| 34 | 4n | TTTT | CCCC | AAAA | 8 | ss3 |
| 35 | 4n | TTTT | CCCC | AAAA | 7.1 | ss3 |
| 36 | 4n | TTTT | CCCC | AAAA | 7.4 | ss3 |
| 37 | 4n | TTTT | CCCC | AAAA | 6 | ss3 |
| 38 | 2n | AA | AA | AA | 0.6 | ss1 |
| 39 | 2n | AA | AA | AA | 0.7 | ss1 |
| 40 | 2n | AA | CC | AA | 0.9 | ss1 |
| 41 | 2n | AA | CC | AA | 1.1 | ss1 |
| 42 | 2n | TT | AA | AA | 1.2 | ss2 |
| 43 | 2n | TT | AA | AA | 1.4 | ss2 |
| 44 | 2n | TT | AA | AA | 1.3 | ss2 |
| 45 | 2n | TT | AA | AA | 1.3 | ss2 |
| 46 | 2n | TT | AA | AA | 1.3 | ss2 |
| 47 | 2n | TT | AA | AA | 1.5 | ss2 |
| 48 | 2n | TT | AA | AA | 1.5 | ss2 |
| 49 | 2n | TT | AA | AA | 1.4 | ss2 |
| 50 | 2n | TT | AA | AA | 1.6 | ss2 |
| 51 | 2n | TT | AA | AA | 1.5 | ss2 |
| 52 | 2n | TT | AA | AA | 1.7 | ss2 |
| 53 | 2n | TT | AA | AA | 1.5 | ss2 |
| 54 | 2n | TT | AA | AA | 1.9 | ss2 |
| 55 | 2n | TT | AA | AA | 2.1 | ss2 |
| 56 | 2n | TT | AA | AA | 1.9 | ss2 |
| 57 | 2n | TT | AA | AA | 2.2 | ss2 |
| 58 | 2n | TT | AA | AA | 2.2 | ss2 |
| 59 | 2n | TT | AA | AA | 1.8 | ss2 |
| 60 | 2n | TT | AA | AA | 2.6 | ss2 |
| 61 | 2n | TT | AA | AA | 2.2 | ss2 |
| 62 | 2n | TT | CA | AA | 2.3 | ss2 |
| 63 | 2n | TT | CA | AA | 2.1 | ss2 |
| 64 | 2n | TT | AA | AA | 3.2 | ss2 |
| 65 | 2n | TT | AA | AA | 2.4 | ss2 |
| 66 | 2n | TT | AA | AA | 2.3 | ss2 |
| 67 | 2n | TT | AA | AA | 2.4 | ss2 |
| 68 | 2n | TT | AA | AA | 2.3 | ss2 |
| 69 | 2n | TT | AA | AA | 2.3 | ss2 |
| 70 | 2n | TT | AA | AA | 2.6 | ss2 |
| 71 | 2n | TT | CC | AA | 3.6 | ss3 |
| 72 | 2n | TT | CC | AA | 2.7 | ss3 |
| 73 | 2n | TT | CC | AA | 4.5 | ss3 |
| 74 | 2n | TT | CC | AA | 4.4 | ss3 |
| 75 | 2n | TT | CC | AA | 5.5 | ss3 |
| 76 | 2n | TT | CC | AA | 5.0 | ss3 |
| 77 | 2n | TT | CC | AA | 5.9 | ss3 |
| 78 | 2n | TT | CC | CC | 6.4 | ss4 |
| 79 | 2n | TT | CC | CC | 10.5 | ss4 |

### Example 2: Assessment of the Spot Allele Dosage in Spotted Watermelons

The present disclosure is based at least in part on the detailed genetic analysis of SNPs and QTLs that are associated with spotted phenotype.

### Materials and Methods

### Samples

Multiple spotted watermelon populations were used for testing. Young leaf samples were obtained from each of the following: (1) a tetraploid F2 population segregating for the spot locus, (2) triploid variety with one spot allele (33%), (3) triploid variety with two spot alleles (67%), (4) triploid variety with three spot alleles (100%), (5) tetraploid line with a present spot locus, (6) tetraploid line with an absent spot locus, (7) diploid line with a present spot locus, and (8) diploid line with an absent spot locus.

### Marker Candidates

A diploid F2 population was used to map the region associated with the spot phenotype. The results obtained from this F2 population were compared with whole genome resequencing of two lines showing the spot phenotype. Fine mapping and recombinant selection was then done on two populations.

The reference genome of ‘Charleston Gray’ (Wu et al. Plant Biotechnol J. 2019 December; 17(12):2246-2258. doi: 10.1111/pbi.13136) was used. The mapping process identified the region linked to the spot phenotype as being between 86340 (start) and 242510 (end) on CG_Chr04 (‘Charleston Gray’ Chromosome 4). In this region, 145 SNPs were identified.

### Phenotypic Assessment of Mapping Population

The diploid F2 population used to map the region was phenotyped for leaf appearance (visual assessment, not quantitative). A pre-grouping of sample plants was done based on spot density.

### High Resolution Melting Analysis

An amplicon of 196 bp was generated by PCR on genomic DNA following standard setup. Post amplification analysis by high-resolution melting curve was performed following manufacturer recommended settings for temperature ramping and fluorescence acquisition. The melting curves were then normalized and analyzed using an internal software.

### Results

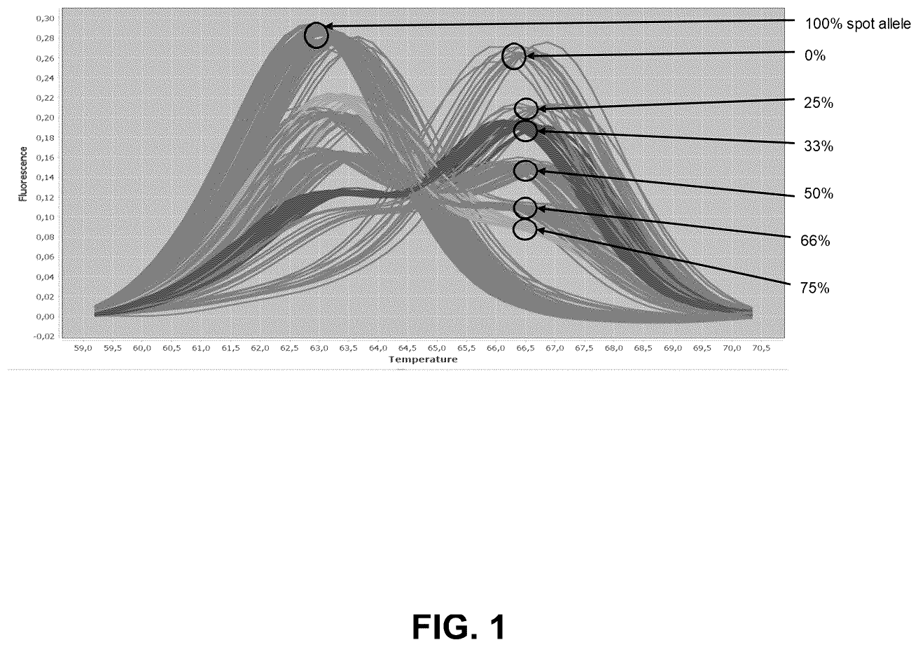

The phenotypic assessment of the F2 diploid mapping population identified some plants with higher leaf spot density (FIGS. 1A-1D), and some plants with lower leaf spot density (FIGS. 1E-1H). Plants with these distinct phenotypes were separated into two groups. Subsequent analysis with markers (see below) confirmed that these phenotypes corresponded to different levels of spotted allele dose. Those plants with lower leaf spot density (FIGS. 1E-1H) were found to be heterozygous, i.e., have 50% spotted allele dose, while those plants with higher leaf spot density (FIGS. 1A-1D) were found to be homozygous, i.e., have 100% spotted allele dose. Without wishing to be bound by theory, it is thought that the allele percentage may correlate with leaf spot density in diploid spotted plants.

Two SNP markers were identified that were able to assess the number of spot alleles in watermelons with different ploidy, which are provided in Table 15, below.

TABLE 15
SNP markers.

| Chromosome | Position | SNP in ‘Charleston Gray’ | SNP in Samples |
|---|---|---|---|
| CG_Chr04 | 134886 | A | T |
| CG_Chr04 | 86340 | A | G |

FIG. 1 shows the results of the high resolution melting analysis conducted using primers and a probe directed to the SNP at position 134886. Each group of curves with a black circle corresponded to a specific percentage of spot allele. Table 16, below, provides the percentages, the number of alleles present, and the corresponding ploidies. For example, 75% spotted was only possible in tetraploid lines, which can have up to four spotted alleles.

TABLE 16
High resolution melting analysis results.

| % Spot | Number of spotted | |
|---|---|---|
| Allele | alleles | Ploidy |
| 100% | Homozygous | All |
| | present | |
| 75% | 3 alleles | Tetraploid (4n) |
| 66% | 2 alleles | Triploid (3n)_ |
| 50% | 2 alleles | Tetraploid (4n) |
| 50% | 1 allele | Diploid (2n) |
| 33% | 1 allele | Triploid (3n) |
| 25% | 1 allele | Tetraploid (4n) |
| 0% | Homozygous absent | All |

These results clearly showed that the identified markers were able to distinguish the allele percentage present in spotted watermelon lines and varieties. Thus, these markers may be used in breeding and/or selection of spotted watermelon lines and varieties.

### Example 3: Phenotypic and Genotypic Ratios for a Segregating Spotted Diploid Watermelon, and Breeding of a Homozygous Spotted Diploid Line (HSDL)

This Example describes the phenotypic and genotypic ratios for a segregating diploid watermelon, as well as the breeding process to obtain a new homozygous spotted diploid line (HSDL).

### Materials and Methods

### Spotted Alleles

Spotted plants were identified by phenotypic assessment. The spotted allele underlying the spotted phenotype was designated as “A”, while the non-spotted allele was designated as “a”.

### Breeding Process

The initial cross was of a spotted plant with a non-spotted plant. The spotted plant was identified by phenotype alone. The F1 generation produced by this cross all had the spotted phenotype. Then, the F1 generation was self pollinated to produce a F2 generation.

### Results

The phenotypes observed in the F2 generation indicated that the F1 generation had the genotype of Aa, i.e., was heterozygous for the spotted allele. There were are four possible combinations of the spotted genotype observed in the F2 population. Table 17 shows the genotypic ratios, while Table 18 shows the phenotypic ratios.

TABLE 17
Genotypic ratios in F2 population.

| | A | a |
|---|---|---|
| A | AA | Aa |
| a | Aa | aa |

TABLE 18
Phenotypic ratios in F2 population.

| | A | a |
|---|---|---|
| A | Spotted (100%) | Spotted (50%) |
| a | Spotted (50%) | Not spotted (0%) |

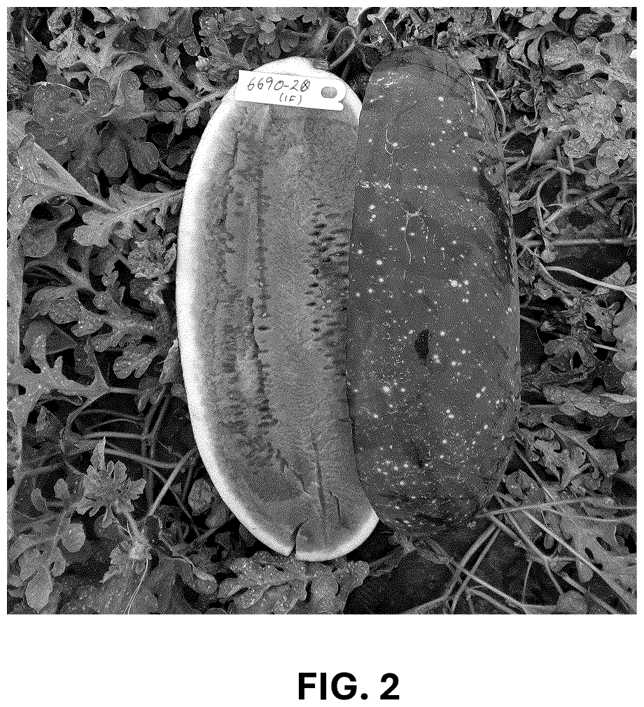

As can be seen from Table 18, the phenotype of the F2 population was 3:1 spotted, with two different percentages of spotted allele (100% and 50%). The F2 plants with the spotted phenotype were then self pollinated and selected for the spotted phenotype for a sufficient number of generations to produce a new homozygous spotted diploid line (HSDL). FIG. 2 shows an exemplary diploid watermelon line with 100% spot allele.

The markers identified in Example 1 can be used to facilitate the breeding process described in this Example. Specifically, the markers for the spot allele are very helpful to use at the F2 stage in order to select the material with the higher dosage of spotted alleles, namely 100% (AA). The use of these markers will allow the production of a diploid line homozygous for the spotted trait (HSDL) in fewer generations of inbreeding.

### Example 4: Genotypic Ratios for a Segregating Tetraploid Watermelon, and Breeding of a Homozygous Spotted Tetraploid Line (HSTL)

This Example describes the phenotypic and genotypic ratios for a segregating tetraploid watermelon, as well as the breeding process to obtain a new homozygous spotted tetraploid line (HSTL).

### Materials and Methods

### Spotted Alleles

Spotted plants were identified by phenotypic assessment. The spotted allele was designated as “A”, while the non-spotted allele was designated as “a”.

### Breeding Process

The initial cross was of a spotted plant with a non-spotted plant. The spotted plant was identified by phenotype alone. The F1 generation produced by this cross all had the spotted phenotype. Then, the F1 generation was self pollinated to produce a F2 generation.

### Results

The phenotypes observed in the F2 generation indicated that the F1 generation had the genotype of AAaa, i.e., was heterozygous for the spotted allele. There were thirty-six possible combinations of the spotted genotype observed in the F2 population. Table 19 shows the genotypic ratios, while Table 20 shows the phenotypic ratios.

TABLE 19
Genotypic ratios in F2 population.

| | AA | Aa | Aa | Aa | Aa | aa |
|---|---|---|---|---|---|---|
| AA | AAAA | AAAa | AAAa | AAAa | AAAa | AAaa |
| Aa | AAAa | AAaa | AAaa | AAaa | AAaa | Aaaa |
| Aa | AAAa | AAaa | AAaa | AAaa | AAaa | Aaaa |
| Aa | AAAa | AAaa | AAaa | AAaa | AAaa | Aaaa |
| Aa | AAAa | AAaa | AAaa | AAaa | AAaa | Aaaa |
| aa | AAaa | Aaaa | Aaaa | Aaaa | Aaaa | aaaa |

TABLE 20
Phenotypic ratios in F2 population.

| | AA | Aa | Aa | Aa | Aa | aa |
|---|---|---|---|---|---|---|
| AA | Spotted | Spotted | Spotted | Spotted | Spotted | Spotted |
| | (100%) | (75%) | (75%) | (75%) | (75%) | (50%) |
| Aa | Spotted | Spotted | Spotted | Spotted | Spotted | Spotted |
| | (75%) | (50%) | (50%) | (50%) | (50%) | (25%) |
| Aa | Spotted | Spotted | Spotted | Spotted | Spotted | Spotted |
| | (75%) | (50%) | (50%) | (50%) | (50%) | (25%) |
| Aa | Spotted | Spotted | Spotted | Spotted | Spotted | Spotted |
| | (75%) | (50%) | (50%) | (50%) | (50%) | (25%) |
| Aa | Spotted | Spotted | Spotted | Spotted | Spotted | Spotted |
| | (75%) | (50%) | (50%) | (50%) | (50%) | (25%) |
| aa | Spotted | Spotted | Spotted | Spotted | Spotted | Not spotted |
| | (50%) | (25%) | (25%) | (25%) | (25%) | (0%) |

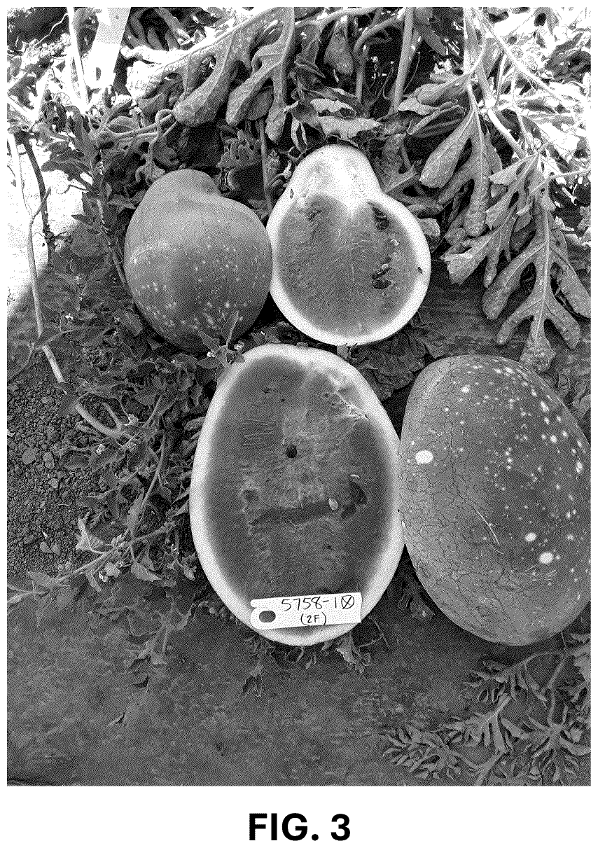

As can be seen from Table 20, the phenotype of the F2 population was 35:1 spotted, with four different percentages of spotted allele (100%, 75%, 50%, and 25%). The F2 plants with the spotted phenotype were then self pollinated and selected for the spotted phenotype for a sufficient number of generations to produce a homozygous spotted tetraploid line (HSTL). FIG. 3 shows an exemplary tetraploid watermelon line with 100% spot allele.

The markers identified in Example 2 can be used to facilitate the breeding process described in this Example. Specifically, the markers for the spot allele are very helpful to use at the F2 stage in order to select the material with the higher dosage of spotted alleles, namely 100% (AAAA). The use of the marker allows the production of a tetraploid line homozygous for the spotted trait (HSTL) in fewer generations of inbreeding.

### OTHER EMBODIMENTS

It will be appreciated that the scope of the present disclosure is to be defined by that which may be understood from the disclosure and claims rather than by the specific embodiments that have been presented by way of example. Elements described with respect to one aspect or embodiment of the present disclosure are also contemplated with respect to other aspects or embodiments of the present disclosure. Moreover, recitation of claim elements in connection with a particular independent claim support recitation of such elements in connection with other independent claims. Throughout the disclosure and claims, where compositions or methods are described as having, including, or comprising specific elements, compositions that consist essentially of, consist of, or do not comprise the recited elements are likewise hereby disclosed. All references cited herein are hereby incorporated by reference.

1. A watermelon seed, wherein the watermelon seed:
(a) comprises at least one spotted phenotype QTL associated with a T allele of SNP 134886 and/or a G allele of SNP 86340; and
(b) has:
(i) a seed size of “ss1,” “ss2,” “ss3,” or “ss2-3”, or
(ii) a 100 SdW between 0.28 g and 10.1 g.

2-4. (canceled)

5. The watermelon seed of claim 1, wherein the 100 SdW is between
1.2 g and 10.1 g.

6. The watermelon seed of claim 1, wherein the watermelon seed is triploid.

7. A watermelon plant produced from the watermelon seed of claim 6.

8. A watermelon fruit produced from the plant of claim 7, wherein the watermelon fruit has a spotted phenotype.

9. The watermelon fruit of claim 8, wherein the watermelon fruit has a 33% spotted phenotype.

10. The watermelon seed of claim 1, wherein the watermelon seed comprises in its genome:
(a) at least one seed size QTL on chromosome 2 associated with a T allele of SNP 34078247; and
(b) at least one seed size QTL on chromosome 6 associated with an A allele of SNP 5640046, a T allele of SNP 5655752, an A allele of SNP 5828256, and/or a G allele of SNP 5828333.

11. The watermelon seed of claim 10, wherein the watermelon seed is homozygous for:
(a) a seed size QTL on chromosome 2 associated with a T allele of SNP 34078247; and
(b) a seed size QTL on chromosome 6 associated with an A allele of SNP 5640046, a T allele of SNP 5655752, an A allele of SNP 5828256, and/or a G allele of SNP 5828333.

12. The watermelon seed of claim 1, wherein the watermelon seed comprises in its genome:
(a) at least one T allele of SNP 34078247; and
(b) at least one of an A allele of SNP 5640046, a T allele of SNP 5655752, an A allele of SNP 5828256, and a G allele of SNP 5828333.

13. The watermelon seed of claim 12, wherein the watermelon seed is homozygous for:
(a) a T allele of SNP 34078247; and
(b) at least one of an A allele of SNP 5640046, a T allele of SNP 5655752, an A allele of SNP 5828256, and a G allele of SNP 5828333.

14-17. (canceled)

18. A method of producing a triploid watermelon seed, the method comprising crossing:
(a) a watermelon plant of claim 34, wherein the watermelon plant is a tetraploid female parent watermelon plant:
(i) comprising at least one spotted phenotype QTL associated with a T allele of SNP 134886 and/or a G allele of SNP 86340; and/or
(ii) that produces seeds having (a) a seed size of “ss1,” “ss2,” “ss3,” or “ss2-3”, or (b) a 100 SdW of 0.28 to 10.1 g, and
(b) a diploid male parent watermelon plant.

19-24. (canceled)

25. The method of claim 18, wherein the method further comprises growing a triploid seed produced by the cross, thereby producing a triploid watermelon plant.

26. The method of claim 25, wherein the triploid watermelon plant produces seedless watermelon fruit having a spotted phenotype.

27-34. (canceled)

35. A watermelon plant, or part thereof, wherein the watermelon plant or part thereof comprises:
(a) at least one T allele of SNP 34078247; and
(b) at least one of an A allele of SNP 5640046, a T allele of SNP 5655752, an A allele of SNP 5828256, and a G allele of SNP 5828333.

36. The watermelon plant, or part thereof, of claim 35, wherein the watermelon plant or part thereof is homozygous for:
(a) a T allele of SNP 34078247; and
(b) at least one of an A allele of SNP 5640046, a T allele of SNP 5655752, an A allele of SNP 5828256, and a G allele of SNP 5828333, and
wherein the watermelon plant has a spotted phenotype.

37-39. (canceled)

40. The watermelon plant, or part thereof, of claim 35, wherein the watermelon plant or part thereof is diploid and has a diploid genotype set forth in any one of Tables 6A-6AB for SNP 34078247, SNP 5640046, and SNP 5828256.

41. The watermelon plant, or part thereof, of claim 35, wherein the watermelon plant or part thereof is tetraploid and has a tetraploid genotype set forth in any one of Tables 6A-6AB for SNP 34078247, SNP 5640046, and SNP 5828256.

42-45. (canceled)

46. The watermelon plant, or part thereof, of claim 35, wherein the watermelon plant or part thereof is triploid and has a triploid genotype set forth in any one of Tables 6A-6AB for SNP 34078247, SNP 5640046, and SNP 5828256.

47-74. (canceled)

75. A method of producing a triploid watermelon fruit, the method comprising crossing a triploid watermelon plant with a diploid watermelon plant,
wherein the diploid watermelon plant is characterized in that has a spotted phenotype and/or at least one spotted phenotype QTL associated with a T allele of SNP 134886 and/or a G allele of SNP 86340.

76-134. (canceled)

135. The watermelon seed of claim 1, wherein the 100 SdW is between 4.4 g and 7.2 g.

136. The watermelon seed of claim 1, wherein the watermelon seed is diploid.
